(19) United States
(12) Patent Application Publication
(10) Pub. No.: US 2006/0099256 A1
(43) Pub. Date: May 11, 2006

(54) Microspheres and related processes and pharmaceutical compositions

(76) Inventors: James C. Price, Athens, GA (US); Wasfy M. Obeidat, Irbid (JO)

Correspondence Address: COLEMAN SUDOL SAPONE, P.C., 714 COLORADO AVENUE, BRIDGE PORT, CT, 06605-1601, US

(21) Appl. No.: 10/538,859

(22) PCT Filed: Nov. 24, 2003
(86) PCT No.: PCT/US03/37744

§ 371 Date: Jun. 14, 2005

Related U.S. Application Data
(60) Provisional application No. 60/429,500, filed on Nov. 27, 2002.

(51) Int. Cl. A61K 9/26

(52) U.S. Cl. 424/469

(57) ABSTRACT

The instant invention provides microspheres and related processes and pharmaceutical compositions useful in the controlled delivery of a wide variety of active ingredients. In one embodiment, the microspheres comprise an active ingredient dispersed within a polymeric composition comprising a first pH insensitive hydrophobic polymer and second pH sensitive hydrophobic polymer, wherein the microspheres, in an aqueous environment having a pH of around 5 or greater, release the active ingredient in a substantially zero-order profile. In another embodiment, the microspheres comprise an active ingredient dispersed within a polymeric composition comprising a first pH insensitive hydrophobic polymer and second water-swellable polymer, wherein the microspheres, in an aqueous environment, release the active ingredient in a substantially zero-order profile. In both of these embodiments, the microspheres are prepared by a non-aqueous emulsion solvent evaporation method.

### FIELD OF THE INVENTION

The instant invention provides microspheres and related processes and pharmaceutical compositions useful in the controlled delivery of a wide variety of active ingredients. In one enteric embodiment, the microspheres comprise an active ingredient dispersed within a polymeric composition comprising a first pH insensitive hydrophobic polymer and second pH sensitive hydrophobic polymer, wherein the microspheres, in an aqueous environment having a pH of around 5 or greater, release the active ingredient in a substantially zero-order profile. In another embodiment, the microspheres comprise an active ingredient dispersed within a polymeric composition comprising a first pH insensitive hydrophobic polymer and second water-swellable polymer, wherein the microspheres, in an aqueous environment, release the active ingredient in a substantially zero-order profile. In both of these embodiments, the microspheres are prepared by a non-aqueous emulsion solvent evaporation method.

### BACKGROUND OF THE INVENTION

Many drugs irritate the stomach, are destroyed by gastric juices, or are not well absorbed in the stomach. Consequently, delivery of such drugs in the small intestine is preferred. Site-specific release of drugs in the small intestine, however, poses unique controlled delivery problems. In order to deliver a drug in useful form and quantity by the oral route to the small intestine, a dosage form must pass through the stomach without releasing a significant amount of drug. In adults, the small intestine extends from duodenum to ileum and is 3.5-6 m long. The pH of the gastrointestinal tract (GI) tract gradually increases as one moves down the GI tract from the stomach (pH 1.5-3) to the early parts of the small intestine, the duodenum (pH 6.5-7.6) to the distal part of the small intestine, the ileum (pH 6.9-7.9). The transit through the GI tract is highly variable and depends on many factors like the fasted/fed state of the subject, quality and quantity of food, size and density of the dosage form, concomitant administration of other drugs and physical exercise. Gastric transit of single unit non-disintegrating dosage forms has been reported to vary from 15 minutes to more than 3 hours. L. C. Kaus, et al., On the intestinal transit of a single nondisintegrating object, Int. J. Pharm., 14, 143-148, 1984. The small intestinal residence time is fairly constant at 3-4 hours.

Irrespective of the preferred site of drug delivery, controlled release drug dosage forms are known to have many advantages over conventional dosing. It is well known that patient compliance is better when the drug dosing is only once or twice daily. It has been reported that, as the number of doses per day increases, there is a greater risk that the patient will either forget or neglect to take every dose. B. Malahy, The effect of instruction and labeling on the number of medication errors made by the patient at home, American Journal of Hospital Pharmacy, 32, 867-859, 1966. Other major advantages are the optimization of drug concentration in plasma and reduction of side effects, particularly for drugs with low therapeutic indexes. For oral administration, advantages of multiple-unit products include ready distribution over a large area, less variable release and release which is less dependent on gastric transit time V. D. Vilivalam, et al., Development and evaluation of controlled-release diclofenac microspheres and tableted microspheres, J. Micro-encapsulation, 11, 455-470, 1994 (“Vilivalam”). This potentially improves drug absorption and reduces local irritation to the GI mucosa. Li S. P., et al., Recent advances in microencapsulation technology and equipment. Drug Dev. Ind. Pharm., 14, 353-376, 1988.

Microspheres are recognized as an effective method to achieve a sustained release effect. Vilivalam. Matrix microspheres can be prepared for many drugs and are very rugged. H. A. M. Sayed, J. C. Price, Tablet properties and dissolution characteristics of compressed cellulose acetate butyrate microcapsules containing succinyl sulfathiazole. Drug Dev. Ind. Pharm., 12, 577-587, 1986. A widely used method to prepare matrix microspheres is emulsion-solvent evaporation. O'Donnell, et al., Preparation of microspheres by the solvent evaporation technique, Advanced Drug Delivery Reviews, 28, 25-42, 1997; K. Suzuki, J. C. Price, Microencapsulation and dissolution properties of a neuroleptic in a biodegradable polymer, poly (dl-lactide), J. Pharm. Sci., 74, 1, 21-24 1985. The emulsion solvent evaporation method offers several advantages. For example, it is a simple process, pH adjustment is not required, the process can be carried out at low or moderate temperatures, and reactive agents or catalysts are not needed. In this method, a dispersion of drug in polymer solution is emulsified in a liquid that is immiscible with the polymer solution. The drug is encapsulated inside the polymer droplet. A. Luzzi, J. Microencapsulation, J. Pharm. Sci., 59, 1367-1376, 1970. Microsphere characteristics are greatly affected by processing and formulation variables. Variables such as stirring speed, drug solubility, solvent type, temperature, morphology and drug loading may be varied in the process. R. Jalil, et al., Biodegradable poly(lactic acid) and poly(lactide-co-glycolide) microcapsules: problems associated with preparative techniques and release properties, J. Microencapsulation, 7, 297-325, 1990, A. J. Shukla, J. C. Price, Effect of drug loading and molecular weight of cellulose acetate propionate on the release characteristics of theophylline microspheres. Pharm. Res., 8, 1396-1400, 1991.

There is a continuing need for improved controlled-release pharmaceutical compositions comprising microspheres that achieve predictable (ideally zero order) delivery of a wide variety of active ingredients in both acidic environments such as the stomach and in environments such as the small intestine where pH can exceed 6. Ideally, such compositions would facilitate the delivery of active ingredients which have a low therapeutic index (e.g., theophylline, in which concentration in the blood should be maintained in the range 10-20 μg/ml). And preferably, such compositions would utilize controlled-release vehicles such as microspheres which are readily manufactured and which are well-suited to meet to all of the aforementioned pharmacological objectives.

### OBJECTS OF THE INVENTION

It is an object of the present invention to provide controlled-release pharmaceutical compositions that achieve predictable (ideally zero order) delivery of a wide variety of active ingredients in both acidic environments such as the stomach and in environments such as the small intestine where pH can exceed 6.

It is a further object of the present invention to provide microspheres that are useful in controlled-release pharmaceutical compositions and that achieve predictable (ideally zero order) delivery of a wide variety of active ingredients in both acidic environments such as the stomach and in environments such as the small intestine where pH can exceed 6.

It is a further object of the present invention to provide microspheres that achieve predictable (ideally zero order) delivery of active ingredients with a low therapeutic index in both acidic environments such as the stomach and in environments such as the small intestine where pH can exceed 6.

It is a further object of the present invention to provide microspheres that are compatible with the physiocochemical properties of a wide variety of drugs, including drug dose size, drug solubility, gastrointestinal stability and pKa.

It is a still further object of the present invention to provide methods of making microspheres that are useful in controlled-release pharmaceutical compositions and that achieve predictable (ideally zero order) delivery of a wide variety of active ingredients in both acidic environments such as the stomach and in environments such as the small intestine where pH can exceed 6.

### SUMMARY OF THE INVENTION

In accordance with the above stated objects, the instant invention provides microspheres that are useful in controlled-release pharmaceutical compositions and that achieve substantially zero order delivery of a wide variety of active ingredients in both acidic environments such as the stomach and in environments such as the small intestine where pH can exceed 6. These microspheres prove particularly useful in delivering active ingredients such as theophylline which have a low therapeutic index. Significantly, in one embodiment, the microspheres, upon dissolution in an aqueous environment having a pH of 6 or greater, substantially deliver all of their active ingredient in about 12 to 24 hours.

Microspheres of the instant invention may be formulated to deliver an extremely broad range of pharmaceutically active ingredients. The microspheres are especially useful for delivery of moderately non-polar active ingredients. However, the microspheres can be formulated to deliver very soluble polar compounds and non-polar, non-soluble compounds by adjusting microsphere composition to slow dissolution (in the case of polar active compounds) or increase solubility (in the case of non-polar active compounds).

In one enteric embodiment of the instant invention particularly useful for delivering active ingredient in the small intestine, the invention provides microspheres comprising an active ingredient dispersed within a polymeric composition comprising a first pH insensitive hydrophobic polymer and second pH sensitive hydrophobic polymer, wherein the microspheres, in an aqueous environment having a pH of around 5 or greater, release the active ingredient in a substantially zero-order profile, and wherein:

(a) the microspheres are formed by a non-aqueous emulsion solvent evaporation method in which the first and second polymers and active ingredient are dispersed in an organic solvent to form a polymer solution phase, the polymer solution phase is emulsified into a second continuous phase comprising a second solvent and a surfactant to form an emulsified dispersion system, and the emulsified dispersion system is agitated and organic solvent evaporated there from to form the microspheres;

(b) the concentration of the second polymer as a percentage of total polymer in the polymer solution phase ranges from around 1% to 35% and total polymer concentration in the polymer solution phase ranges from around 5% to around 35%;

(c) microsphere particle diameter ranges from approximately 25 μm to approximately 1,000 μm;

(d) the weight percentage of active ingredient in a microsphere ranges from around 5% to around 50%; and

(e) active ingredient concentration is highest in the microsphere core.

In another embodiment, the invention provides microspheres comprising an active ingredient dispersed within a polymeric composition comprising a first pH insensitive hydrophobic polymer and second water-swellable polymer, wherein the microspheres, in an aqueous environment, release the active ingredient in a substantially zero-order profile, and wherein:

(a) the microspheres are formed by a non-aqueous emulsion solvent evaporation method in which the first and second polymers and active ingredient are dispersed in an organic solvent to form a polymer solution phase, the polymer solution phase is emulsified into a second continuous phase comprising a second liquid having limited solvent ability for the components of the polymer solution phase and a surfactant to form an emulsified dispersion system, and the emulsified dispersion system is agitated and organic solvent evaporated there from to form the microspheres;

(b) the concentration of the second polymer as a percentage of total polymer in the polymer solution phase ranges from around 0.25% to 10%, total polymer concentration in the polymer solution phase ranges from around 5% to around 35% (more preferably around 5% to 20%), and the viscosity of the polymer solution phase ranges from around 20 cps to around 1000 cps, more preferably about 50 to about 300 cps;

(c) microsphere particle diameter ranges from approximately 25 μm to approximately 1,000 μm;

(d) the weight percentage of active ingredient in a microsphere ranges from around 5% to around 50%; and

(e) active ingredient concentration is highest in the microsphere core.

Enteric microspheres of the instant invention are made by a non-aqueous solvent evaporation method comprising:

(a) dispersing a first pH insensitive hydrophobic polymer, a second pH-sensitive hydrophobic polymer, and an active ingredient in an organic solvent to form a polymer solution phase;

(b) emulsifying the the polymer solution phase into a second continuous phase comprising a second liquid having limited solvent ability for the components of the polymer phase and a surfactant to form an emulsified dispersion system; and

(c) agitating the emulsified dispersion system and evaporating the organic solvent therefrom to form the microspheres

wherein (1) the concentration of the second polymer as a percentage of total polymer in the polymer solution phase ranges from around 0.25% to 10% and total polymer concentration in the polymer solution phase ranges from around 5% to around 35% (2) microsphere particle diameter ranges from approximately 25 μm to approximately 1,000 μm (3) the weight percentage of active ingredient in a microsphere ranges from around 5% to around 50%, and (4) active ingredient concentration is highest in the microsphere core. All polymers, solvents, and ingredient used in the microspheres and related processes of the instant invention are biocompatible.

Microspheres of the instant invention are also made by a non-aqueous solvent evaporation method comprising:

(a) dispersing a first pH insensitive hydrophobic polymer, a second water-swellable polymer, and an active ingredient in an organic solvent to form a polymer solution phase;

(b) emulsifying the the polymer solution phase into a second continuous phase comprising a second solvent and a surfactant to form an emulsified dispersion system; and

(c) agitating the emulsified dispersion system and evaporating the organic solvent there from to form the microspheres

wherein (1) the concentration of the second polymer as a percentage of total polymer in the polymer solution phase ranges from around 0.25% to 10%, total polymer concentration in the polymer solution phase ranges from around 5% to around 35% (more preferably around 5% to around 20%), and the viscosity of the polymer solution phase ranges from around 20 cps to around 1000 cps, more preferably around 50 cps to 300 cps,

(2) microsphere particle diameter ranges from approximately 25 μm to approximately 1,000 μm (preferably, at least 75 μm within this range), (3) the weight percentage of active ingredient in a microsphere ranges from around 5% to around 50%, preferably around 20% to 40%; and (4) active ingredient concentration is highest in the microsphere core.

In preferred enteric embodiments of the instant invention, the first and second polymers are selected from the group consisting of cellulose acetate, cellulose acetate propionate, cellulose acetate butyrate, cellulose acetate phthalate, cellulose propionate butyrate, and combinations and mixtures thereof. In particularly preferred embodiments, the first polymer is cellulose acetate butyrate (CAB) and the second polymer is cellulose acetate phthalate (CAP), the total concentration of CAB in the polymer solution phase is between around 7% to around 9%, and the total concentration of CAP in the polymer solution phase as a percentage of total polymer is between around 1% to 3%.

In other preferred embodiments of the instant invention, the first polymer is selected from the group consisting of cellulose acetate, cellulose acetate propionate, cellulose acetate butyrate, cellulose propionate butyrate, and combinations and mixtures thereof, and the second polymer is selected from the group consisting of a low-substituted cellulose ether or internally cross-linked cellulose derivatives of sodium carboxymethylcellulose, hydroxypropyl-methylcellulose (HPMC), a hydroxypropylcellulose (HPC), a poly(ethylene oxide), a hydroxy-ethylcellulose, or a hydrogel forming polymer.

In particularly preferred embodiments, the first polymer is cellulose acetate butyrate (CAB) and the second polymer is hydroxypropylcellulose (HPC), the total concentration of CAB in the polymer solution phase is between around 7% to around 9%, and the total concentration of HPC in the polymer solution phase as a percentage of total polymer is between around 0.5% to 3%.

Most preferably, microspheres of the present invention are about 300 microns (μm.) in size. Even more preferably, the microspheres are between 150 μm and 200 μm in size. However, the microspheres of the present invention may be formulated to achieve virtually any size less than 300 μm by adjusting agitation rates, adjusting viscosity of the emulsified dispersion phase, or increasing the temperature used during formulation.

These and other features of the instant invention are described further in the following detailed description.

### BRIEF DESCRIPTION OF THE DRAWINGS

FIG. 1 illustrates a log probability plot of particle size distributions of CAB-CAP microspheres of the instant invention.

FIGS. 2-10 illustrate theophylline release profiles in HCl for different size CAB-CAP microspheres, or varying CAB:CAP ratio microspheres, of the instant invention.

FIG. 11 illustrates Higuchi plots for different size fractions for CAB-CAP microspheres of the instant invention.

FIG. 12 illustrates theophylline release profiles in phosphate buffer (pH 7.5) for CAB-CAP microspheres of the instant invention.

FIG. 13 illustrates a log probability plot of particle size distributions of CAB-HPC microspheres of the instant invention.

FIGS. 14-19 illustrate theophylline release profiles in simulated intestinal fluid for different size CAB-HPC microspheres, or varying CAB:HPC ratio microspheres, of the instant invention.

FIGS. 20 and 21 illustrate theophylline release profiles for different size CAB-HPC microspheres of the instant invention compared to those of CAB microspheres.

### DETAILED DESCRIPTION OF THE INVENTION

As used herein, the following terms have the following respective meanings.

“Biocompatible”: the polymers, solvents and other agents of the invention must be biocompatible; that is they must not cause irritation or necrosis in the environment of use. The environment of use is a fluid environment and may comprise a subcutaneous or intramuscular portion or body cavity of a human or animal.

“Enteric” as used herein means a composition comprising an active ingredient having an increased resistance to degradation in the upper gastrointestinal tract, and/or a decrease in the release or exposure of active ingredient in the upper gastrointestinal tract.

A “microsphere” as used herein means a “matrix microsphere” in which active ingredient particles are dispersed in direct contact with the polymer matrix. Such a microsphere is a homogeneous or monolithic particle in which the drug is dissolved or dispersed throughout the polymer matrix. Release of drugs from a microsphere is a mass transport phenomenon involving diffusion of drug molecules from the region of high concentration in the dosage form to a region of low concentration in the surrounding environment. Mathematical release rate expressions describing microspheres are provided in T. Higuchi, Mechanism of sustained action medication, Theoretical analysis of rate of release of solid drug dispersed in solid matrices. J. Pharm. Sci., 52.

An “Emulsion-solvent evaporation process” or “non-aqueous emulsion-solvent evaporation process” can be used to make microspheres and involves dissolving or dispersing a drug in solution of one or more polymers, e.g. a mixture of CAB and CAP, in single or mixed organic solvent having low boiling point to form a polymer solution phase. The phase is then emulsified into a continuous immiscible phase containing a low concentration of colloid or surfactant to stabilize the emulsion formed. Reduced pressure or heat is often applied to evaporate off the organic solvent and the microspheres are collected by filtration or centrifugation.

Emulsion-based processes used herein involve the preparation of two separate phases: a first phase (referred to herein as the “polymer phase” and also known as the “solvent” or “W” phase), which consists of a dispersion or solution of an active agent in a solution of polymer dissolved in a first solvent, and a second phase (the “continuous dispersion medium (O-phase)”), which consists of a solution of surfactant and a second liquid (e.g., light or heavy mineral oil) that is at least partially immiscible with the first solvent of the dispersed phase. After the first and second phases are prepared, they are combined using dynamic or static mixing to form an emulsion (also referred to herein as the “emulsified dispersion system”), in which microdroplets of the first phase are dispersed in the second phase. The microdroplets then are hardened to form polymeric microspheres that contain the active agent. The hardening step is carried out by removal of the first solvent from the microdroplets, in the case of the instant invention by evaporation.

It is known that several variables may be varied, or must be considered, in employing an emulsion-solvent evaporation process. These include drug/polymer ratio, the nature of the surfactant and organic phase volume and their effect on the final product. Increasing the drug/polymer ratio can decrease microsphere yield; a reduction in the amount of organic solvent (internal phase) can cause an increase in microsphere diameter along with a lowering of release rate. Immiscibility/miscibility characteristics of the solvent with the oily phase plays an important role in microsphere formation; where ethyl cellulose or similar polymers are used, microcapsule or microsphere formation mainly depends on solvent diffusion rate into the oily phase and on drug solubility in the ethyl cellulose solvent. Variables such as stirring speed, drug solubility, solvent type, temperature, morphology and drug loading have been reported frequently as being very important in making microspheres by the process.

“Emulsify” means to form a stable dispersion of one liquid in a second immiscible liquid. An example of such would be milk. An “immiscible” liquid is a liquid which is not soluble in another substance or liquid, for example, oil in water. In contrast, two substances that are mutually soluble in all proportions are is said to be miscible.

“Surfactants” or “emulsifiers” that can be used in the instant invention include but are not limited to anionic surfactants, nonionic surfactants, polyoxyethylene-castor oil derivatives, polyvinylpyrrolidone, polyvinyl alcohol, carboxymethylcellulose, lecithin, and sorbitan sesquioleate. Sorbitan sesquioleate (for example, Span 83 and Arlacel 83) and magnesium stearate are preferred surfactants.

“Hydrophobic polymers” are polymers that are substantially insoluble in water and that dissolve in organic solvents. As used herein, the term includes pH insensitive polymers and pH-sensitive polymers. “Hydrophobic”, “pH insensitive” and “pH sensitive” as used herein are relative terms and in making microspheres of the instant invention, the hydrophobicity and pH-sensitivity of each polymer when compared to the other(s) is determinative. “pH insensitive” implies that pH has a limited effect on polymer dissolution at a pH of around 5 or greater. “pH sensitive” implies that pH has a more pronounced effect on polymer dissolution at a pH of around 5 or 6 or greater than would be the case with a “pH insensitive” polymer. “Hydrophobic polymers” include cross-linked polyvinyl alcohol, polyolefins or polyvinyl chlorides; regenerated, insoluble, non-erodible cellulose, acylated cellulose, esterified celluloses, cellulose acetate propionate, cellulose acetate butyrate, cellulose acetate phthalate, cellulose acetate diethyl-aminoacetate; polyurethanes, polycarbonates, and microporous polymers formed by co-precipitation of a polycation and a polyanion modified insoluble collagen.

Exemplary esterified or acylated cellulose derivatives suitable for use in the instant invention as pH sensitive or pH insensitive hydrophobic polymers include those which are substituted by one to three acetyl groups or by one or two acetyl groups and a further acyl radical other than acetyl, such as cellulose acetate dimethylamino acetate, cellulose acetate ethyl and methyl carbonate, cellulose acetate phthalate, cellulose acetate succinate, cellulose acetate chloroacetate, cellulose diacetate, cellulose triacetate, cellulose acetate ethyl oxalate, cellulose acetate trimellitate, hydroxypropyl methylcellulose phthalate, cellulose acetate methyl and butyl sulfonate, cellulose acetate octate, cellulose acetate laurate, cellulose acetate p-toluene sulfonate, cellulose acetate ethyl and methyl carbamate, cellulose acetate valerate, cellulose acetate maleate, and the like, and combinations and mixtures thereof.

Preferred cellulose esters are cellulose acetate, cellulose acetate propionate, cellulose acetate butyrate, cellulose acetate phthalate, cellulose propionate butyrate, and the like, and combinations and mixtures thereof. Such cellulose derivatives can be prepared by any known technique in the art (for example, see Kirk-Othmer, Encyclopedia of Chemical Technology, 3rd Edition, Vol. 5, John Wiley & Sons, New York, N.Y., 1979, p. 89-129; and Libscomb, A. G., Cellulose Acetate: Its Manufacture and Applications, Ernest Benn, Ltd. London, GB, 1933), or can be obtained commercially (for example, from Eastman Chemical Products, Inc., Kingsport, Tenn.).

Particularly preferred cellulose esters that do not exhibit pH-dependent solubility characteristics and act as pH insensitive hydrophobic polymers in the instant invention include cellulose acetate butyrates (CAB). Particularly preferred cellulose acetate butyrates are CAB-171-15PG, CAB-381-0.1, CAB-381-20, CAB-500-5, and CAB-553-0.4, which are all commercially available from Eastman Chemical Products, Inc., Kingsport, Tenn.). In the foregoing description, the first two digits indicate the approximate butyryl content at the triester stage, the third digit indicates the number of hydroxyl groups for each four anhydroglucose units, and the last digit(s) indicate the viscosity of the ester. CABs with higher butyryl content, such as greater than 30%, tend to be more effective in suppressing crystal formation. Particularly preferred cellulose esters that do exhibit relative pH-dependent solubility characteristics at pH's of around 5 or 6 or greater and can function as pH sensitive hydrophobic polymers include cellulose acetate phthalate (CAP). Those of ordinary skill in the art will be able to select suitable combinations or mixtures of the aforementioned pH insensitive and pH sensitive hydrophobic polymers for use in making microspheres of the instant invention.

“Water-swellable polymers” includes a hydroxypropyl-methylcellulose (HPMC), a hydroxypropylcellulose (HPC), a poly(ethylene oxide), a hydroxyethylcellulose or a combination thereof. Water-swellable polymers also include a low-substituted cellulose ether or internally cross-linked cellulose derivatives of sodium carboxymethylcellulose. An especially preferred type of HPMC for use in accordance with the invention is an HPMC sold under the trademark Methocel (Dow Chemical Co.) or equivalents. Suitable Methocels include the K grades such as Methocel K15M, Methocel K100M, Methocel K100LV and Methocel K4M. Other suitable Methocels include the E, F and J grades. An especially preferred type of HPC for use in accordance with the invention is an HPC sold under the trademark Klucel (Hercules, Inc.) or equivalents. Suitable Klucels include Klucel LF, Klucel JF, Klucel GF, Klucel MF and Klucel HF. An especially preferred type of poly(ethylene oxide) for use in accordance with the invention is a poly(ethylene oxide) sold under the trademark Sentry Polyox (Union Carbide Corp.) or equivalents. Suitable Polyoxs include the Polyox WSR grades such as Polyox WSR Coagulant, Polyox WSR-301, Polyox WSR-303, Polyox WSR N-12K, Polyox WSR N-60K, Polyox WSR-1105, Polyox WSR-205 and Polyox WSR N-3000.

Water-swellable polymers also include polymers that form hydrogels. A hydrogel is defined as a substance formed when an organic polymer (natural or synthetic) is cross-linked via covalent, ionic, or hydrogen bonds to create a three-dimensional open-lattice structure which entraps water molecules to form a gel. Naturally occurring and synthetic hydrogel forming polymers, polymer mixtures and copolymers may be utilized as hydrogel precursors. Examples of materials which can be used to form a hydrogel include polysaccharides such as alginate and modified alginates, synthetic polymers such as polyphosphazines, and polyacrylates, which are crosslinked ionically, or block copolymers such as Pluronics or Tetronics.™, polyethylene oxide-polypropylene glycol block copolymers which are crosslinked by temperature or pH, respectively. Other materials include proteins such as fibrin, polymers such as polyvinylpyrrolidone.

“Organic solvent” as used herein includes halogenated hydrocarbons (e.g., dichloromethane, chloroform, carbon tetrachloride, etc.), alcohols (e.g., ethanol, methanol, etc.), acetonitrile, and acetone. These solvents can also be used as a mixture. The preferred organic solvents are acetonitrile, and acetone. Acetone is particularly preferred.

“Non-polar” as used herein means a composition that has lipophilic groups or has sufficient lipophilic character such that the composition is soluble in a hydrocarbon phase and “polar” as used herein means a composition that has hydrophilic groups soluble in an aqueous phase.

The surfactant-containing “second solvent” used in the instant invention may be a substance belonging to any of the categories of polymers, mineral oils or vegetable oils, which are not miscible with the “organic solvent” defined previously, and in which the hydrophobic or water-swellable polymers are not appreciably soluble. Typical examples are silicone oil, sesame oil, soybean oil, corn oil, cottonseed oil, coconut oil, linseed oil, mineral oil, n-hexane, n-heptane, and mixtures thereof.

“Controlled release” generally refers to compositions, e.g., pharmaceutically acceptable carriers, for controlling the release of an active agent or drug incorporated therein, typically by slowing the release of the active agent or drug in order to prevent immediate release. Such controlled release compositions and/or carriers are used herein to prolong or sustain the release of an active agent or drug incorporated.

The phrase “substantially zero-order” as used herein means delivery of an active agent at a release rate which is approximately constant once steady state is attained, typically within 12 hours or less after dissolution in an aqueous environment. While variability in blood levels of active agent are contemplated within the scope of this meaning once steady state release is attained, the depletion rate of active agent over the duration of use should typically not exceed about 10% per hour for oral controlled release dosage forms but may be much slower for implantable or transdermal dosage forms.

“Therapeutic index”: Toxicity and therapeutic efficacy of pharmaceutical compositions employing microspheres as described herein can be determined by standard pharmaceutical procedures in cell cultures or experimental animals, e.g., for determining the LD 50 (the dose lethal to 50% of the population) and the ED 50 (the dose therapeutically effective in 50% of the population). The dose ratio between toxic and therapeutic effects is the “therapeutic index” and it can be expressed as the ratio between LD 50 and ED 50. Compounds which exhibit high therapeutic indices are preferred. The data obtained from these cell culture assays and animal studies can be used in formulating a range of dosage for use in humans. The dosage of such compounds lies preferably within a range of circulating concentrations that include the ED 50 with little or no toxicity. The dosage may vary within this range depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics,” Ch. 1 p. 1).

“Pharmaceutical compositions” comprising microspheres of the present invention may be formulated in a conventional manner using one or more pharmaceutically acceptable carriers. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polylactic acid, polyglycolic acid, polyethylene glycol, sodium carboxymethylcellulose, polyacrylates, waxes, polyethylene-polyoxypropylene-block polymers, polyethylene glycol, sucrose, lactose and wool fat.

Pharmaceutical compositions comprising microspheres of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally, or intravenously.

Sterile injectable forms of pharmaceutical compositions comprising microspheres of the present invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as cetyl alcohol or similar a similar alcohol.

Pharmaceutical compositions comprising microspheres of the present invention may be orally administered in any orally acceptable dosage form including, but not limited to, capsules, tablets, aqueous suspensions or solutions. In the case of tablets for oral use, carriers which are commonly used include lactose, sucrose, microcrystalline cellulose and corn starch. Lubricating agents, such as magnesium stearate and stearic acid, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, pharmaceutical compositions comprising microspheres of the present invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, certain mono-, di-, and triglycerides of long chain fatty acids and polyethylene glycols.

Pharmaceutical compositions comprising microspheres of the present invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, pharmaceutical compositions comprising microspheres of the present invention may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, stearyl alcohol, 2-octyldodecanol, benzyl alcohol and water.

For ophthalmic use, pharmaceutical compositions comprising microspheres of the present invention may be formulated from very fine microspheres as suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

Pharmaceutical compositions comprising microspheres of the present invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of active ingredient used in pharmaceutical compositions comprising microspheres of the present invention will vary depending upon the host treated, the particular mode of administration.

The term “active ingredient” (or “agent,” “drug,” “medicament” and “pharmaceutical”) is intended to have the broadest meaning and includes at least one of any therapeutic, prophylactic, pharmacological or physiological active substance, cosmetic and personal care preparations, and mixtures thereof, which is delivered to a mammal to produce a desired, usually beneficial, effect. More specifically, any active agent that is capable of producing a pharmacological response, localized or systemic, irrespective of whether therapeutic, diagnostic, cosmetic or prophylactic in nature, is within the contemplation of the invention. It should be noted that the active agents can be used singularly or in combinations and mixtures.

There is no limitation on the type of active ingredient that can be used in this invention. However, slightly non-polar active agents are preferred. In particular, the microspheres of the instant invention are useful in delivering active ingredients such as theophylline which have a low therapeutic index. (Theophylline concentration in the blood should be maintained in the range 10-20 μg/ml.)

The active agents contained in the carrier composition can be in different forms depending on the solubility and release characteristics desired, for example as neutral molecules, components of molecular complexes, and pharmaceutically acceptable salts, free acids or bases, or quaternary salts of the same. Simple derivatives of the drugs such as pharmaceutically acceptable ethers, esters, amides and the like which have desirable retention and release characteristics but which are easily metabolized at body pH, and enzymes, pro-active forms, pro-drugs and the like, can also be employed.

Steroidal hormones and active agents that generally tend to be poorly soluble or insoluble can be used in the microspheres of the instant invention and include, for example, Estrogenically effective steroid hormones such as Colpormon, Conjugated Estrogens, Estradiol (17.beta.- and .alpha.-) and its Esters (e.g., Acetate, Benzoate, Cypionate, Dipropionate Diacetate, Enanthate, Undecylate and Valerate), Estriol, Estrone, Ethinyl Estradiol, Equilenin, Equilin, Mestranol, Moxestrol, Mytatrienediol, Quinestradiol and Quinestrol; Progestagenically effective steroid hormones such as Allylestrenol, Anagestone, Chlormadinone Acetate, Delmadinone Acetate, Demegestone, Desogestrel, 3-Keto Desogestrel, Dimethisterone, Dydrogesterone, Ethinylestrenol, Ethisterone, Ethynodiol (and Diacetate), Flurogestone Acetate, Gestodene, Gestonorone Caproate, Haloprogesterone, (17-Hydroxy- and 17-Acetate-) 16-Methylene-Progesterone, 17.alpha.-Hydroxyprogesterone (Acetate and Caproate), Levonorgestrel, Lynestrenol, Medrogestone, Medroxyprogesterone (and Acetate), Megestrol Acetate, Melengestrol, Norethindrone (Acetate and Enanthate), Norethisterone, Norethynodrel, Norgesterone, Norgestimate, Norgestrel, Norgestrienone, 19-Norprogesterone, Norvinisterone, Pentagestrone, Progesterone, Promegestone, Quingestrone and Trengestone; Androgenically effective steroid hormones such as Aldosterone, Androsterone, Boldenone, Cloxotestosterone, Dehydroepiandrosterone, Fluoxymesterone, Mestanolone, Mesterolone, Methandrostenolone, Methyltestosterone, 17.alpha.-Methyltesteosterone, 17.alpha.-Methyltestosterone 3-Cyclopentyl Enol Ether, Norethandrolone, Normethandrone, Oxandrolone, Oxymesterone, Oxymetholone, Prasterone, Stanlolone, Stanozolol, Testosterone (Acetate, Enanthate, Isobutyrate, Propionate and Undecanoate), Testosterone 17-Chloral Hemiacetal, Testosterone 17.beta.-Cypionate and Tiomesterone.

Other specific drugs which can be used in microspheres of the instant invention include:

1. Alpha-Adrenergic Agonist agents such as Phenylpropanolamine and Talipexole.

2. Analgesics and/or Anti-Migraine such as Acetaminophen, Acetylsalicylic Acid, Buprenorphine, Codeine, Fentanyl, Hydomorphone, Lisuride, Salicylic Acid derivatives, Sufentanil and Sumatriptan.

3. Anti-Allergic agents such as Amlexanox, Astemizole, Azelastine, Cromolyn, Fenpiprane, Ibudilast, Nedocromil, Oxatomide, Pentigetide, Repirinast, Tranilast and Traxanox.

4. Anesthetic agents such as Benzocaine, Bupivicaine, Cocaine, Dibucaine, Dyclonine, Etidocaine, Lidocaine, Mepivacaine, Prilocaine, Procaine and Tetracaine.

5. Anoretic agents such as Fenfluramine, Mazindol and Phentermine.

6. Anti-Bacterial (antibiotic) agents including Aminoglycosides, B-Lactams, Cephamycins, Macrolides, Penicillins, Polypeptides and Tetracyclines.

7. Anti-Cancer agents such as Aminolevulinic Acid, 5-Fluouracil, Methotrexate, Tamoxifen and Taxol.

8. Anti-Cholinergic agents such as Atropine, Eucatropine and Procyclidine.

9. Anti-Diabetic agents such as Glipizide, Glyburide, Glypinamide, Insulins, Repaglinide, Rosiglitazone and Troglitazone.

10. Anti-Emetic agents such as Acetylleucine Monoethanolamine, Alizapride, Benzquinamide, Bietanautine, Bromopride, Buclizine, Chlorpromazine, Clebopride, Cyclizine, Dimenhydrinate, Dipheniodol, Domperidone, Granisetron, Meclizine, Methalltal, Metoclopramide, Metopimazine, Nabilone, Ondansteron, Oxypendyl, Pipamazine, Piprinhydrinate, Prochlorperazine, Scopolamine, Tetrahydrocannabinols, Thiethylperazine, Thioproperzaine, Trimethobenzamide and Tropisetron.

11. Anti-Fungal agents such as Clortrimazole, Ketoconazole, Miconazole, Nystatin and Triacetin.

12. Antihistamine agents such as Tricyclics such as Ahistan, Etymemazine, Fenethazine, N-Hydroxyethylpromethazine Chloride, Isopromethazine, Mequitazine, Promethazine, Pyrathiazine, and Thiazinamium Methyl Sulfate, and Loratadine and Clobenzepam.

13. Anti-Hyperlipoproteinemic agents such as Atorvastatin, Cerivastatin, Lovastatin, Pravastatin and Simvastatin.

14. Anti-Hyperthyroid agents such as Methimazole.

15. Anti-Inflammatory and/or Corticoid agents such as Beclomethasone, Betamethasone (and Acetate, Diproprionate and Valerate), Corticosterone, Cortisone, Deoxycortocosterone (and Acetate), Dexamethasone, Diclofenac, Fenoprofen, Flucinolone (and Acetonide), Fludrocortisone, Fluocinonide, Flunisolide, Fluradrenolide, Flurbiprofen, Halcinonide, Hydrocortisone (and Acetate), Ibuprofen, Ibuproxam, Indoprofen, Ketoprofen, Ketorolac, Naproxen, Oxametacine, Oxyphenbutazone, Piroxicam, Prednisolone, Prednisone, Suprofen and Triamcinolone (and Acetonide).

16. Anti-Malarial agents such as Pyrimethamine.

17. Anti-Parkinson's and/or Anti-Alzhiemer's agents such as Biperiden, Bromocriptine, Cabergoline, 1-Hydroxy-Tacrine, Levodopa, Lisuride, Pergolide, Pramipexole, Quinpirole, Ropinirole, Rivastigmine, Physostigimine, Selegiline (Deprenyl and L-Deprenyl), Tacrine and Teruride.

18. Anti-Psychotic and/or Anti-Anxiety and/or Anti-Depressant agents such as Acetophenazine, Bromperidol, Chlorproethazine, Chlorpromazine, Clomipramine, Clozapine, Fluoxetine, Fluphenazine, Haloperidol, Loxapine, Mesoridazine, Molindone, Paroxetine, Perphenazine, Piperacetazine, Sertraline, Thiopropazate, Thioridazine, Thiothixene, Trifluoperazine, Triflupromazine and Venlafaxine.

19. Anti-Ulcerative agents such as Enprostil and Misoprostol.

20. Anti-Viral agents such as Acyclovir, Rimantadine and Vidarabine.

21. Anxiolytic agents such as Azapirones such as Buspirone and Ipsapirone, Benzodiazepines such as Alprazolam, Chlordiazepoxide, Clonazepam, Clorazepate, Diazepam, Flurazepam, Halazepam, Lorazepam, Oxazepam, Oxazolam, Prazepam and Triazolam.

22. B-Adrenergic agonist agents such as Albuterol, Carbuterol, Fenoterol, Metaproterenol, Mirtazapine, Rimiterol, Quinterenol, Salmefamol, Soterenol, Tratoquinol, Terbutaline and Terbuterol.

23. Bronchodilators such as Ephedrine derivatives, Epiniphrine, Isoproterenol, Albuterol, Salbutanol, Clenbuterol and Theophylline.

24. Cardioactive agents such as Atenolol, Benzydroflumethiazide, Bendroflumethiazide, Calcitonin, Captopril, Chlorothiazide, Clonidine, Clopamide, Dobutamine, Dopamine, Diltiazem, Enalapril, Enalaprilat, Gallopamil, Indomethacin, Isosorbide (Dinitrate and Mononitrate), Monoxidil, Nicardipine, Nifedipine, Nitroglycerin, Papaverine, Prazosin, Procainamide, Propranolol, Prostaglandin (E.sub.1 and E.sub.2), Quinidine Sulfate, Timolol, and Verapamil.

25. Central Nervous System stimulants and agents such as Dextroamphetamine, Methylphenidate (and each Enantiomer and Free Base Form) and Nicotine.

26. Cholinergic agents such as Acetylcholine, Arecoline, Bethanechol, Carbachol, Choline, Methacoline, Muscarine and Pilocarpine.

27. Muscle relaxants such as Baclofen.

28. Narcotic antagonist agents such Nalmfene and Naloxone.

An oral enteric controlled release drug delivery system of matrix microspheres with near zero order kinetics was developed in accordance with the instant invention. The experimental details of the preparation and analyses of these microspheres are presented hereinafter in Example 1. Polymer mixtures of a pH sensitive polymer, cellulose acetate phthalate (CAP) and a pH insensitive polymer, cellulose acetate butyrate (CAB381-20) of various ratios were used to obtain the desired constant release rate of theophylline from matrix microspheres. Microspheres with 33% theoretical drug loading of anhydrous micronized theophylline core material were prepared by the emulsion solvent evaporation method. Dissolution studies were conducted with USP dissolution Apparatus II at 37° C. using simulated gastric fluid without enzymes for the first two hours followed by simulated intestinal fluid (SIF) without enzymes for 24 hours. The release mechanism of microspheres in SIF may be similar to a porous reservoir system where the drug is concentrated in the central part of the microspheres. The release kinetics of theophylline from 180-500 μm size range microspheres containing the polymer mixture followed near zero order release kinetics for all formulations that were studied. The microsphere preparations are therefore useful for oral delivery of theophylline and other drugs.

In the embodiment described, CAP polymer is used with CAB381-20 in matrix microspheres prepared by emulsion-solvent evaporation in order to (a) increase theophylline release rates (b) achieve a pH dependent release dosage form, i.e. enteric matrix dosage form, and (c) control the drug release rates so that zero order release kinetics are achieved in matrix microspheres.

Microsphere Preparation

Table 1 shows the different formulas prepared from the polymer mixture CAP and CAB381-20 using an emulsion solvent evaporation method in accordance with the instant invention. The most successful microsphere preparations were those using CAB 381-20 at concentrations 7.5 to 8.5% (w/w) of the total polymer concentration dissolved in acetone and CAP at a ratio of 1-2.5% to that of the total polymer concentration.

Particle Size Distribution

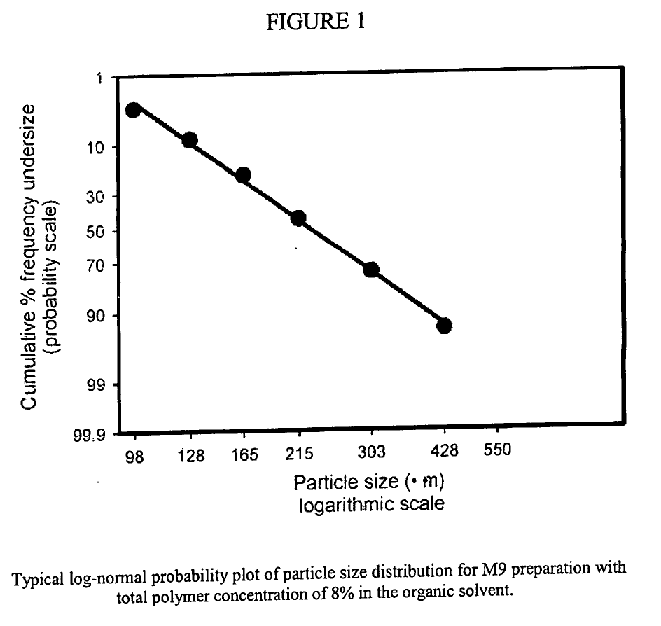

One of the methods used to control the particle size distribution of microspheres prepared by the solvent evaporation method is by altering the agitation intensity during emulsification process. In the present invention, particle size distribution of microspheres prepared with all different polymer mixture solutions can be adjusted by altering the agitation intensity so that each yielded a similar particle size distribution. A typical log-probability plot of size distribution of M9 microspheres with CAP percentage of 1% in a total polymer concentration of 8% is shown in FIG. 1, where the size distribution of microspheres is generally narrow with geometric mean of about 240 μm and a geometric standard deviation of 1.54 calculated from 50% undersize/16% undersize.

Drug Loading

The analysis of drug content of microspheres with a theoretical drug loading of 33.3% calculated from the weight of drug and polymer (the ratio of weights of theophylline: CAB381-20 and CAP was 1:2) varied from 24.0% -24.8% for 180-600 μm size fractions of microspheres. No significant differences in drug loading were found between different microsphere preparations.

Release Studies

Drug release from microspheres composed of a mixture of CAB381-20 and CAP polymers was determined to be related mainly to the dissolution rate of CAP and the integrity of the hydrophobic barrier of CAB381-20. CAP polymer dissolves in the vicinity of pH 6 (24). Its dissolution rate depends on the pH of the microenvironment of the dissolving polymer. It has been reported that the dissociation of CAP polymer in the dissolution fluid is the rate-determining step in its dissolution.

TABLE 1
Microsphere formulas prepared from the polymer mixture CAP and
CAB381-20 polymer mixture using the emulsion-solvent
evaporation method. All formulas contained micronized
anhydrous theophylline to give a theoretical
concentration of 33.3% in the microspheres.

| | | Total polymer |
|---|---|---|
| | Concentration of CAP as | concentration in the polymer |
| Formula | a percentage of the total polymer | solution phase (% W/W) |
| M1 | 25 | 10 |
| M2 | 10 | 10 |
| M5 | 1 | 7.5 |
| M6 | 2 | 7.5 |
| M7 | 5 | 7.5 |
| M8 | 1 | 8 |
| M9 | 2.5 | 8 |
| M10 | 1 | 8.5 |
| M3 | 2.5 | 8.5 |
| M11 | 5 | 8.5 |

The rate of proton transfer is governed by the Bronsted Catalyst Law and the greater the pKa and the concentration of the basic salt used in the buffered dissolution medium, the greater the dissolution rate of CAP. Permeability of a material to water and drugs plays an important role in determining the overall dissolution of CAP microspheres. Enteric materials should be impermeable to the gastric fluid, which has a low pH.

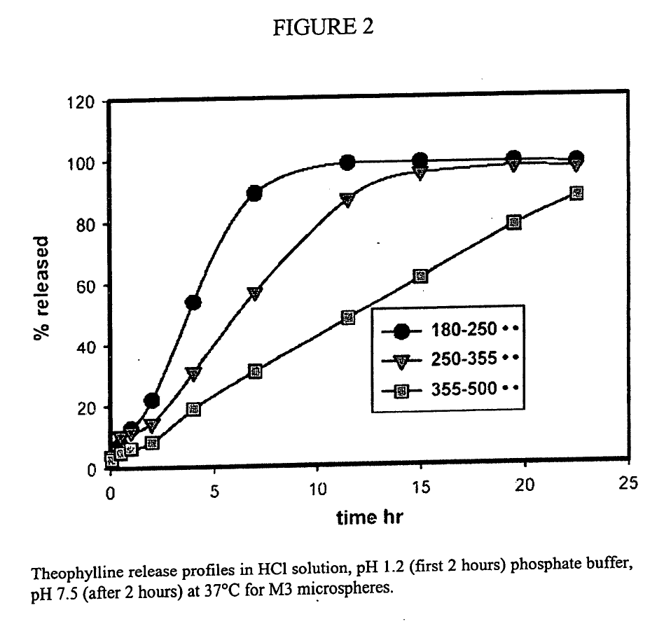

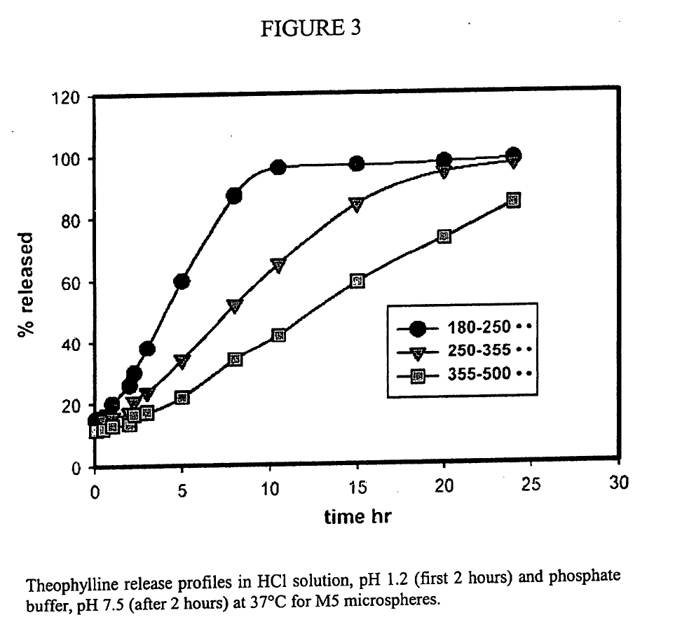

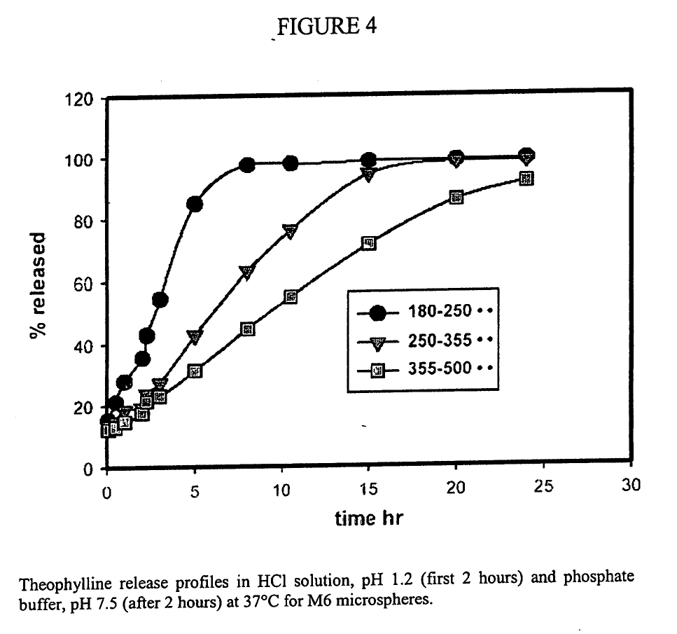

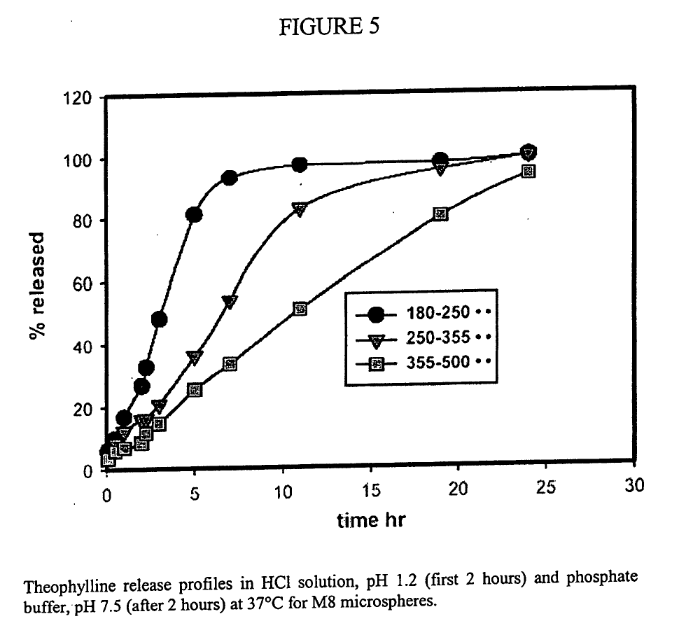

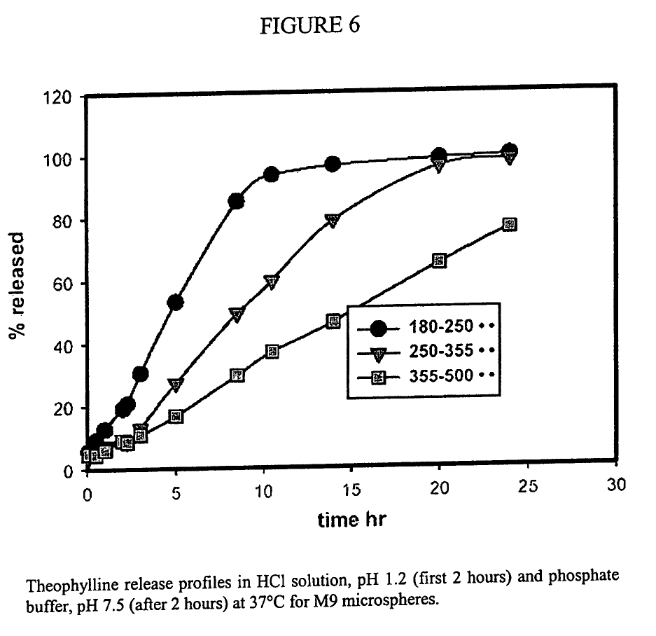

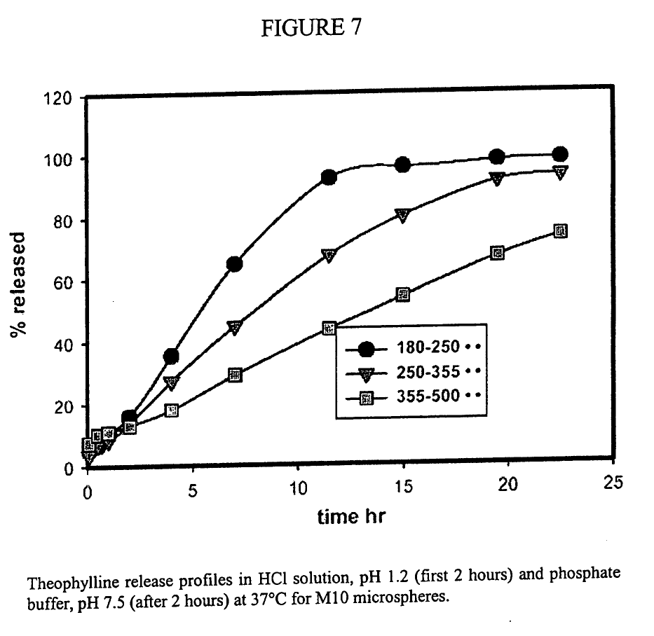

Drug dissolution profiles are shown in FIGS. 2-7 for different size fractions of microspheres of various microsphere preparations. These dissolution profiles can be divided into three stages.

Initial release stage: a small initial drug release was noticed just few minutes after suspending the microspheres in the acid dissolution medium; and higher release rates were associated with smaller size fractions. This may be because, during the initial stage, the drug at or near surface of the microspheres quickly diffused through the buffer-filled pores of the polymer mixture. For the same weight sample, smaller particles have greater surface and therefore have more drug near the surface for the initial rapid release phase.

Release in acid after initial stage: Most of the drug released in the initial stage is due mainly to drug at or near the surface and could be removed with an acid wash prior to the dissolution analysis. After the initial release and during the dissolution in the acidic environment (SGF, pH 1.2, 37° C.) the profile of most formulations flattens out until the media pH is increased. However, a slight increase in the amount of drug released at the end of the acid stage. This behavior of matrix microspheres containing CAP in the acidic media could be explained by the hygroscopic properties of CAP. Higher permeability could be due also to the preparation method. It has been reported that the use of highly volatile solvents in sprayed films yield films with a very high degree of porosity. The use of highly volatile solvents (such as acetone) in microsphere preparation could increase the degree of the porosity of these microspheres containing low viscosity CAP. Also, CAP polymer films, while continuous, can be permeable to ionic solutions and act as a diffusion membrane. Although drug was shown to leach out of microspheres in the acidic region, the rates after the initial release were very low throughout the 2 hour period. This indicates that CAP does not dissolve in the acidic medium, but rather it has some permeability, taking into account that theophylline solubility in both acidic and alkaline media is quite similar due to its neutrality.

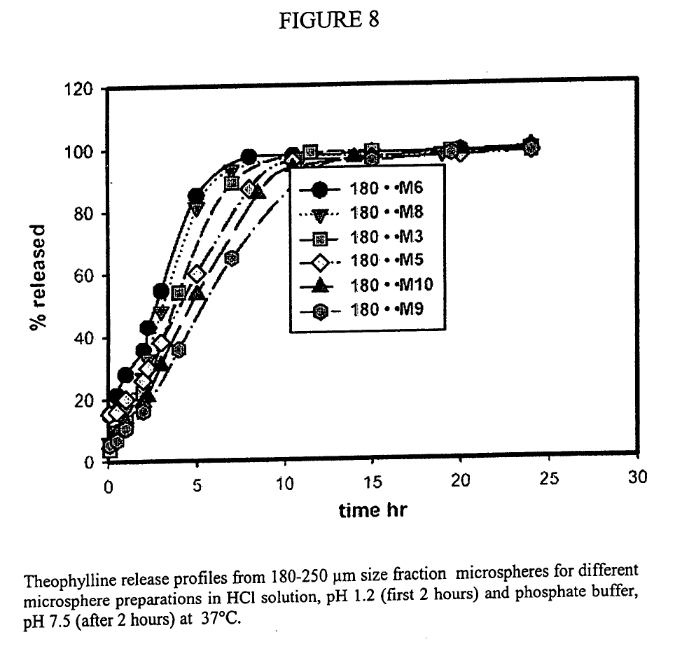

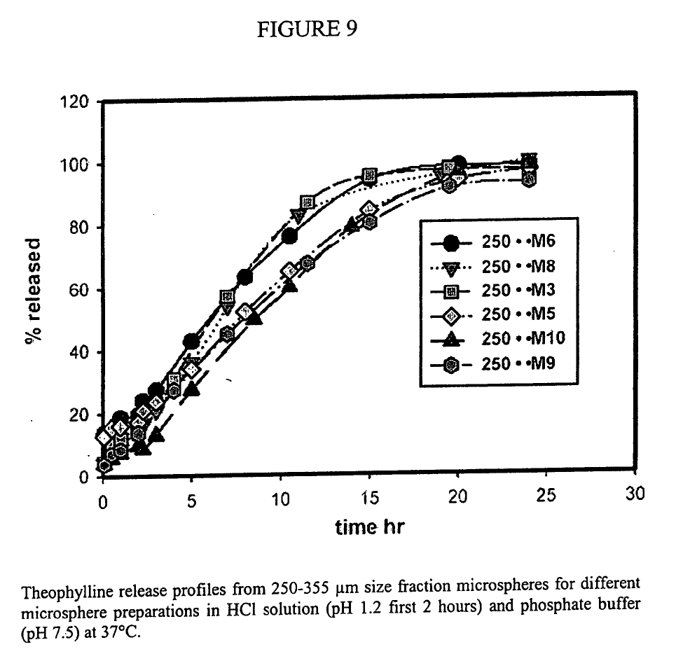

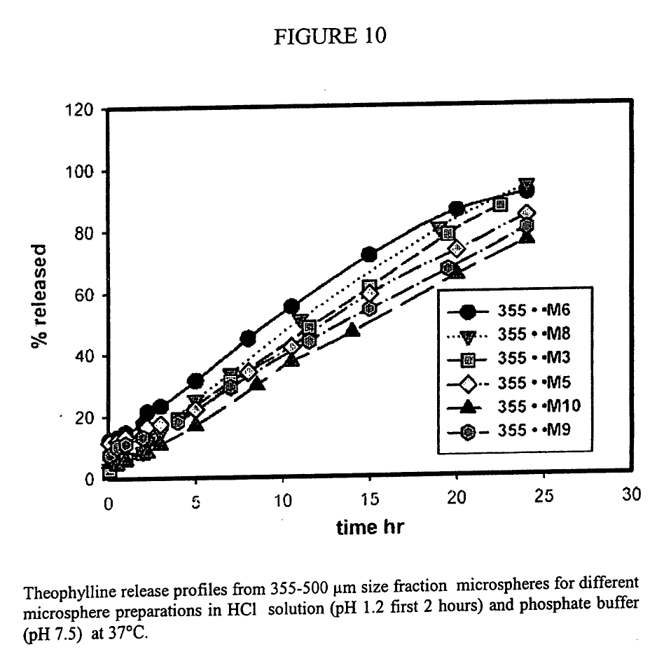

Slightly alkaline stage: During the dissolution in the slightly alkaline-buffered medium (SIF, pH 7.5, 37° C.), higher release rates were noticed for all microsphere size fractions. Slopes of the release profiles in the acidic and the slightly alkaline dissolution media are compared in Tables 2 and 3 where correlation coefficients are also shown. It is evident that slopes are larger in the slightly alkaline media indicating higher theophylline release rates in this medium. Moreover, the release rates are independent of time (zero order kinetics) for all microsphere formulations and size fractions that were studied. Different rates of drug release are observed when comparing formulations containing different ratios of CAP and CAB381-20 as shown in FIGS. 8, 9 and 10. The dissolution profiles at this stage indicate that the CAP polymer at the surfaces and within the outer polymer matrix dissolves quickly leaving behind a porous CAB381-20 membrane. At this time the rate of liquid penetration is controlled by the porosity of the microsphere. As dissolution times increase, more CAP dissolves creating a porous environment for more water to penetrate the microsphere accompanied with more drug diffusion and release. These pores are believed to be small in size and are proportional to the ratio of CAP in the formula, but they are believed to be uniformly distributed throughout the matrix. At the low CAP polymer ratios in the microsphere preparations, release rates are regulated mainly through the porous environment created from CAP leaching out. The porosity created in the polymers allows water to penetrate and drug molecules to leach out.

Microscopy studies indicate that the drug is concentrated toward the central portion of the microspheres thus forming a reservoir of drug for controlled diffusion.

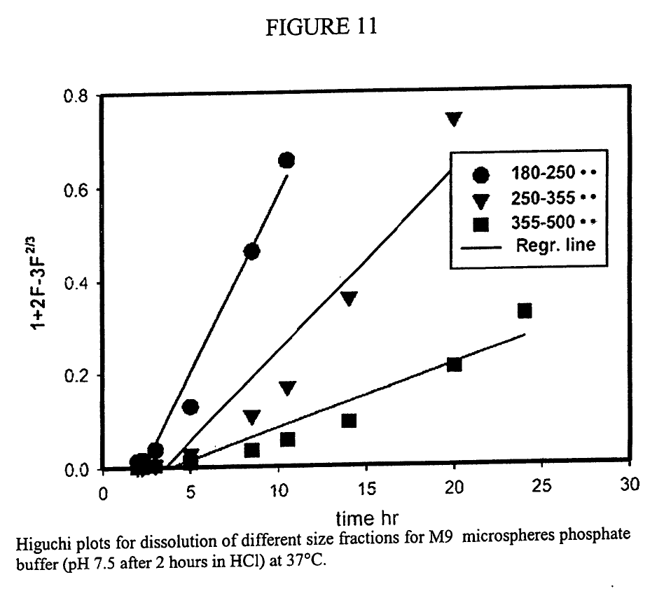

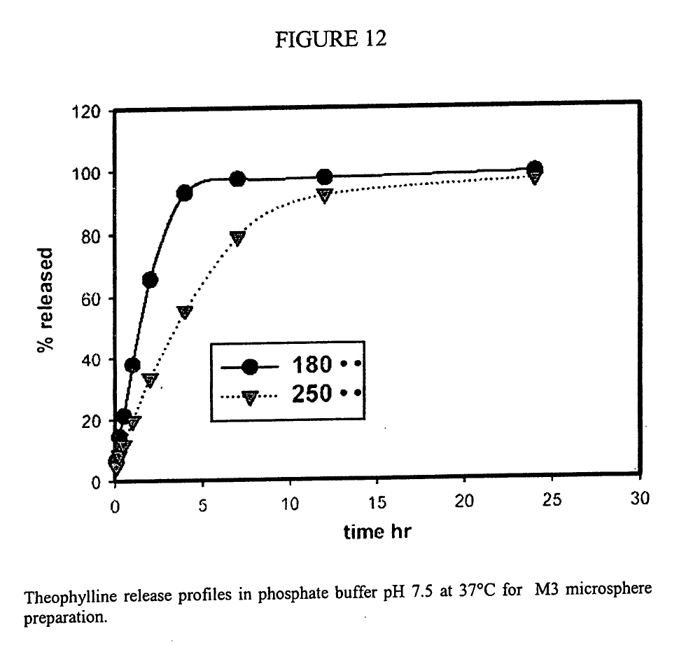

Release kinetics: The release of a drug is usually a diffusion process that depends on the environment of the drug unit. T. Higuchi published equations to describe the release of drug from planar and spherical matrix systems. For most matrix microspheres, the rate of drug release from a granular spherical pellet usually can be described by the Higuchi matrix model, which can be written as shown in the following equation:
1+2F−3F^{2/3}=Kt

Where F is the fraction of drug remaining at time t, K is a combined constant and K=6DCₛVₛₚ/τr₂ for the granular pellet, D is the diffusion coefficient, Cₛ is the solubility of the drug in the matrix or the dissolution fluid in the matrix pores, Vₛₚ is the specific volume of the drug, τ is the tortuosity of the porous system and r is the radius of the microspheres. In this study release profiles did not follow Higuchi spherical matrix release as shown in FIG. 11, instead a near zero order release pattern is seen in most CAB381-20, CAP mixture microspheres until about 80% of drug released. Then, the release rates decreased and reached plateau at about 90% of drug released. The degree of this effect depends to large extent on the ratio of CAP in the microsphere formulation as well as the total polymer concentration or viscosity in the organic phase. The preparations containing CAP could be adjusted to increase release rates as well as to obtain time independent release pattern. Furthermore, dissolution was also conducted for some preparations in a buffer solution of pH 7.5 alone without using the acid media as a first stage. FIG. 12 show the release profile of M3 preparations in phosphate buffer of pH 7.5. It was evident that T50% was low compared to the same preparation tested in the two-stage dissolution (FIG. 2) due to the absence of the acid phase, which is considered as a lag phase. It was also evident that the release profiles do follow near zero order kinetics throughout most of the release times.

TABLE 2
Slopes and correlation coefficients of release profiles of various
microsphere preparations in pH 1.2 medium.
0-2 hours (in SGF)

| | | | Correlation |
|---|---|---|---|
| | | | coefficient for zero |
| Formula | Size | Slope | order mechanism |
| M3 | 180-250 μm | 9.52 | 0.9994 |
| | 355-500 μm | 2.73 | 0.9534 |
| M5 | 180-250 μm | 5.86 | 0.9775 |
| | 355-500 μm | 1.08 | 0.9026 |
| M9 | 180-250 μm | 6.94 | 0.9976 |
| | 355-500 μm | 2.35 | 0.9024 |
| M10 | 180-250 μm | 5.84 | 0.9915 |
| | 355-500 μm | 2.63 | 0.9180 |

TABLE 3
Slopes and correlation coefficients of release profiles of various
microsphere preparations in the pH 7.5 medium
Dissolution in SIF after 2 hours in SGF

| | | | Correlation |
|---|---|---|---|
| | | | coefficient for zero |
| Formula | Size | Slope | order mechanism |
| M3 | 180-250 μm | 13.26 | 0.9921 |
| | 355-500 μm | 3.91 | 0.9980 |
| M5 | 180-250 μm | 11.15 | 0.9989 |
| | 355-500 μm | 3.32 | 0.9967 |
| M9 | 180-250 μm | 10.30 | 0.9962 |
| | 355-500 μm | 3.15 | 0.9980 |
| M10 | 180-250 μm | 9.75 | 1.0000 |
| | 355-500 μm | 3.02 | 0.9971 |

In another embodiment of the instant invention, a water-swelling polymer, hydroxypropylcellulose (HPC), was used in a polymer solution phase containing a hydrophobic polymer, cellulose acetate butyrate (CAB 381-20), to make matrix microspheres containing theophylline. The experimental details of the preparation and analyses of these microspheres are presented hereinafter in Example 2. Release rates and release patterns of CAB381-20 were modified because of the swelling property of HPC in aqueous solutions. Optimization of these microsphere formulations provided a system that had near zero order release kinetics.

Matrix microspheres were prepared by the emulsion solvent evaporation method using Span 83 as an emulsifier. Theophylline (theoretical concentration of 33.3%) was dispersed in different viscosities of polymer mixture solutions of CAB381-20 and HPC in acetone to yield similar drug to polymer ratios. The concentration of HPC to CAB381-20 in these formulas varied from 0.5-5% (w/w) with a total polymer concentration of 7-9% (w/w) in acetone. Dissolution studies were conducted with USP dissolution Apparatus II at 37° C. using simulated intestinal fluid without enzymes as a dissolution medium. More than twelve different formulas were prepared; some showed release patterns useful for controlled release oral dosing. Rapid initial release was noticed with the incorporation of HPC and release rates increased in all formulas as the percentage of HPC increased. This is likely attributed to the property of HPC to swell in contact with water.

Most preparations followed the Higuchi spherical matrix model in their dissolution. However, a few microsphere formulations made of HPC, CAB381-20 polymer mixture showed the ability to release the drug by near zero order kinetics after an initial rapid release. At late stages (after 70-80% release) the kinetics tended to follow the Higuchi spherical matrix model. Incorporating HPC to some extent in a polymer phase containing hydrophobic polymer CAB381-20 in microsphere preparation has a pronounced effect on the release of theophylline from such microspheres. This effect is primarily related to the water-swelling behavior of HPC. Table 4 shows the relative viscosities of different formulations of various proportions of HPC and CAB381-20. It is clear that CAB381-20 is the main ingredient that determines the viscosity of the mixture due to its higher proportions in the formula, while HPC has mild effect at the concentrations employed.

Size Distribution

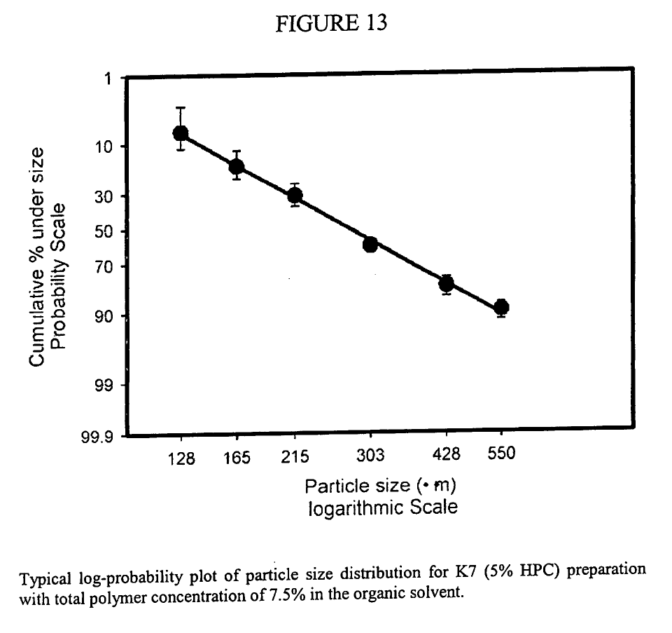

One of the methods used to control the particle size distribution of microspheres prepared by solvent evaporation method is by altering the agitation intensity during emulsification process. It was evident that a change in polymer viscosity has a substantial influence on particle size distribution, with higher viscosities favoring larger particle size. In CAB-HPC embodiments, particle size distribution of microspheres prepared with all different polymer mixture solutions could be adjusted by altering the agitation intensity to yield similar particle size distribution. A typical log-probability plot of size distribution of K7 microspheres preparation with a ratio HPC:CAB381-20 of 2% is shown in FIG. 13, where the size distribution of microspheres is generally narrow with geometric mean of about 280 μm and a geometric standard deviation of 1.7 calculated from 50% undersize/16% undersize.

Drug Loading

The analysis of drug content of microspheres with a theoretical drug loading of 33.3% calculated from the weight of drug and polymer (the ratio of weights of theophylline: CAB381-20 and HPC was 1:2) varied from 24.6-25.8 for 180-600 μm size fractions of microspheres. The average drug loading was higher when HPC was combined with CAB381-20 rather than that of CAB381-20 alone.

Release Studies

Previous studies on microspheres prepared using CAB381-20 at apparent viscosities of (115-240 cps) showed extended release profiles (T50% is 40-60 hours for 180-250 μm microspheres) of the encapsulated theophylline. Matrix microspheres containing combined polymers of different characteristics (CAB381-20 and HPC) were prepared at different ratios, but constant drug to total polymer ratio (theoretical drug concentration is 33.3%). Different formulas were prepared; some as summarized in Table 4.

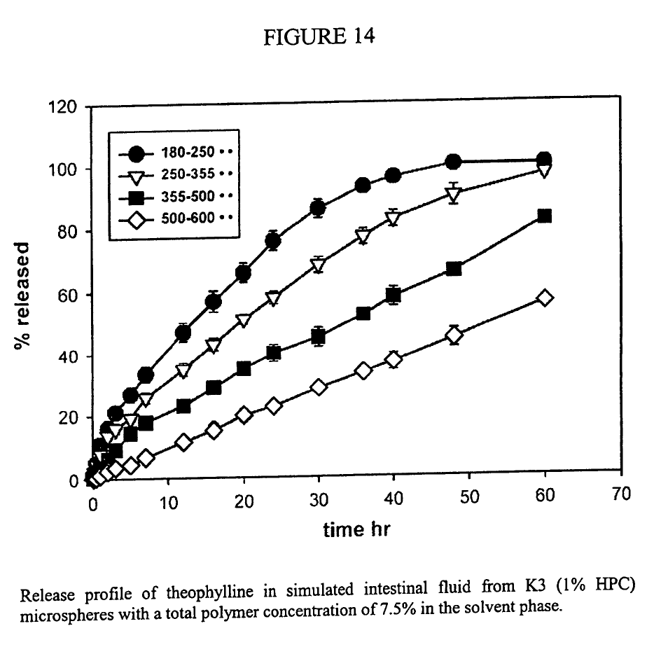

Dissolution studies were conducted for microsphere size fractions of 180 μm, 250 μm, 355 μm and 600 μm for at least 24 hours in simulated intestinal fluid without enzymes. FIGS. 14-16 show the dissolution profiles of K3, K6 and K7 microspheres preparations respectively. It is shown that release rates increased with a decrease in microsphere sizes. Also it is evident from these figures that an increase in HPC ratio is accompanied by a significant increase in theophylline release rates. This is seen more clearly when comparing preparations with fixed total polymer concentration but different HPC concentrations. Compared to CAB381-20 microspheres, T50% has been decreased with HPC being added. Total polymer solution viscosity also affects the magnitude of this decrease.

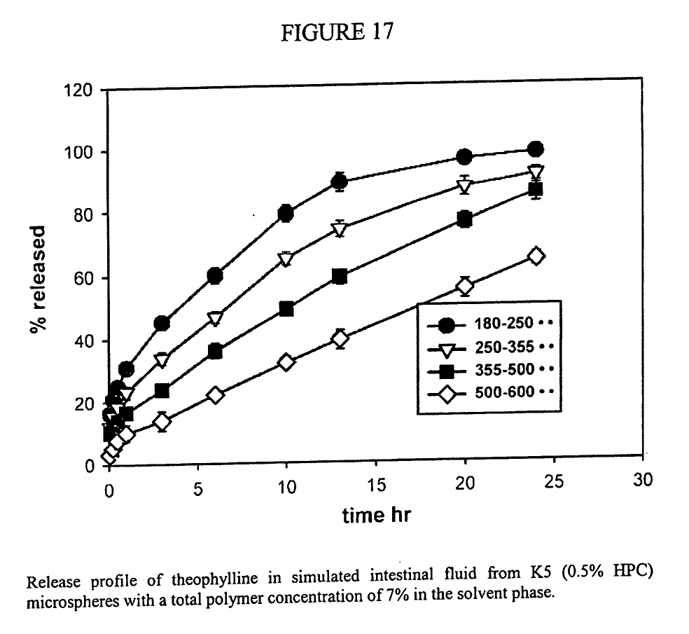

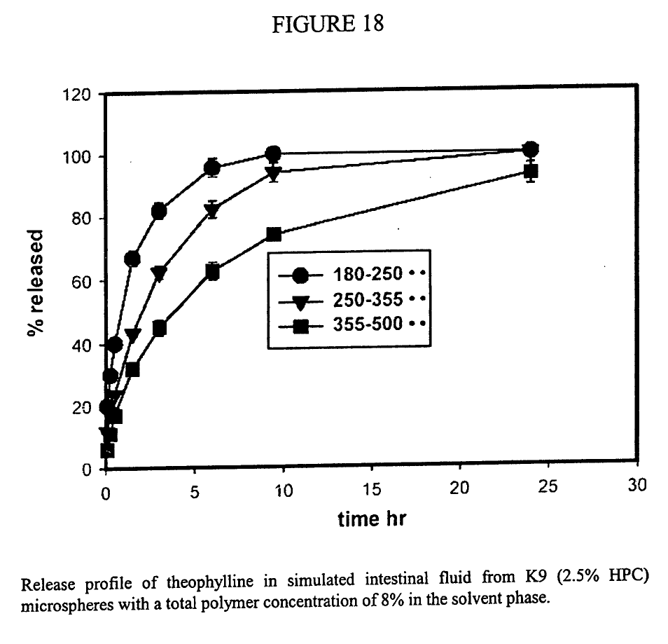

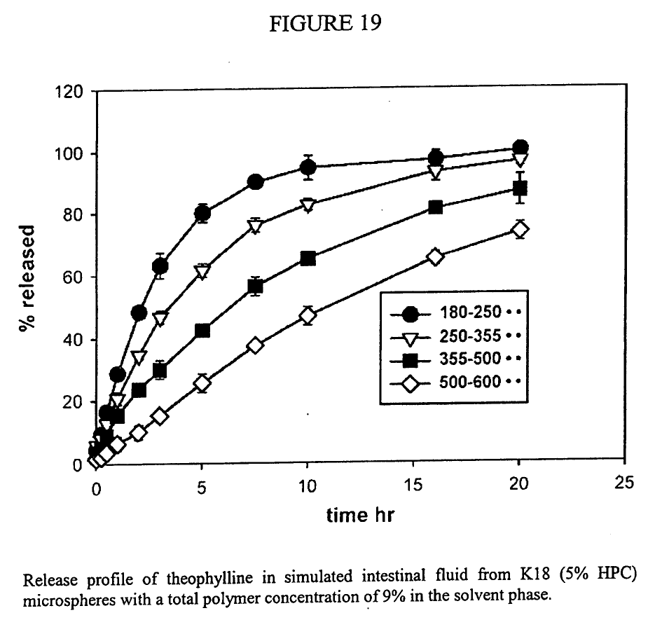

Unlike preparations K6, K3 and K5 which show release profiles near to the zero order pattern after an initial rapid release, most preparations show matrix release which follows the Higuchi spherical matrix model as shown in FIGS. 17-19. These types of release could be explained as follows: When a glassy (or dry) polymer, such as HPC, comes into contact with water or any other medium which it is thermodynamically compatible, the solvent penetrates into the free spaces on the surface between the macromolecular chains. When enough water has entered into the matrix, the glass transition temperature (T_{g}) of the polymer drops to the level of the experimental temperature (which is usually 3720 C. for release studies) except for polymers that the T_{g} is far below experimental temperatures. Therefore polymers with a T_{g} greater than 37° C. in their dry state can be used to prepare swelling controlled release dosage forms.

TABLE 4
Formulas of HPC and CAB381-20 microspheres with corresponding
apparent viscosities. All formulas contained micronized anhydrous
theophylline to give a theoretical concentration of 33.3%
in the microspheres.

| | | Total polymer | |
|---|---|---|---|
| | Concentration of | concentration in the | |
| | HPC as | polymer | Apparent viscosity |
| | a percentage of the | solution phase | of polymer solution |
| Formula | total polymer | (W/W) | phase (cps) |
| K5 | 0.5% | 7% | 88 |
| K13 | 1% | 7% | 81 |
| K3 | 1% | 7.5% | 113 |
| K12 | 1.5% | 7.5% | 109 |
| K6 | 2% | 7.5% | 106 |
| K7 | 5% | 7.5% | 98 |
| K9 | 2.5% | 8% | 140 |
| K15 | 5% | 8% | 130 |
| K14 | 2.5% | 8.5% | 217 |
| K16 | 5% | 8.5% | 203 |
| K17 | 2.5% | 9% | 234 |
| K18 | 5% | 9% | 228 |

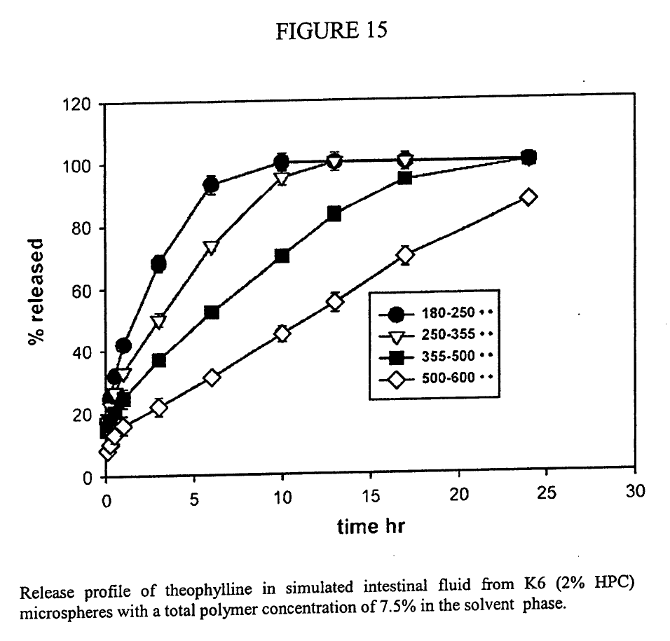

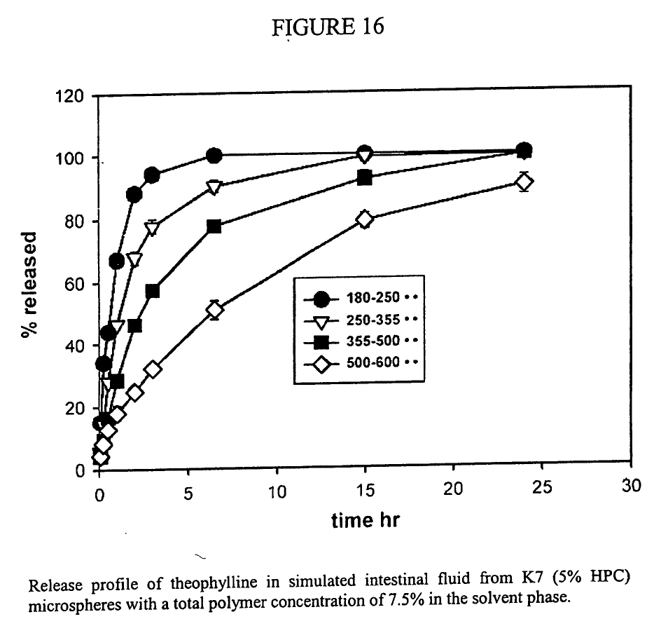

HPC hydrates and swells in aqueous medium forming a gel; the presence of HPC decreases the membrane barrier effect of the CAB381-20 by increasing the water penetration into the microsphere and also by increasing the porosity of the microsphere structure. The degree of this effect depends to large extent on the ratio of HPC in the microsphere formulation as well as the total polymer concentration or viscosity in the organic phase. When the HPC to CAB381-20 ratio is in favor of increased porosity and water penetration, the drug leaches out in a way best described by the Higuchi spherical matrix model (FIGS. 16, 18, and 19). However, in some cases, HPC ratio could be adjusted to increase release rates as well as to obtain time independent release pattern. This could be seen in FIG. 14 (1% HPC in 7.5% total polymer concentration) and FIG. 15 (2% HPC in 7.5% total polymer concentration) where a near zero order release pattern was obtained up to 70-80% for 30 and 10 hours respectively after an initial rapid release.

These release patterns could be explained based on the swelling and relaxation properties of HPC as mentioned earlier in addition to the modification of CAB381-20 microsphere structure as follows: In the initial stage, the liquid (dissolution medium) rapidly dissolves the drug present at the immediate surface of the microspheres. As dissolution times increase, the swollen HPC allows more water to penetrate the microsphere and that is accompanied by countercurrent drug diffusion. Thus in formulas K3 and K6 (FIGS. 14 and 15 respectively) water penetration and hence drug release are regulated by the swollen HPC and the barrier effect of CAB381-20. Therefore, it seems that HPC has disrupted the polymer barrier system exhibited by CAB381-20 microspheres and transformed the system into a pseudo-reservoir structure. This release pattern has been noticed in a few formulations having certain HPC to CAB381-20 ratios with a definite viscosity and this can occur only at very limited combinations of those polymers where there is a balance the effects of both polymers ratios and the total polymer viscosity.

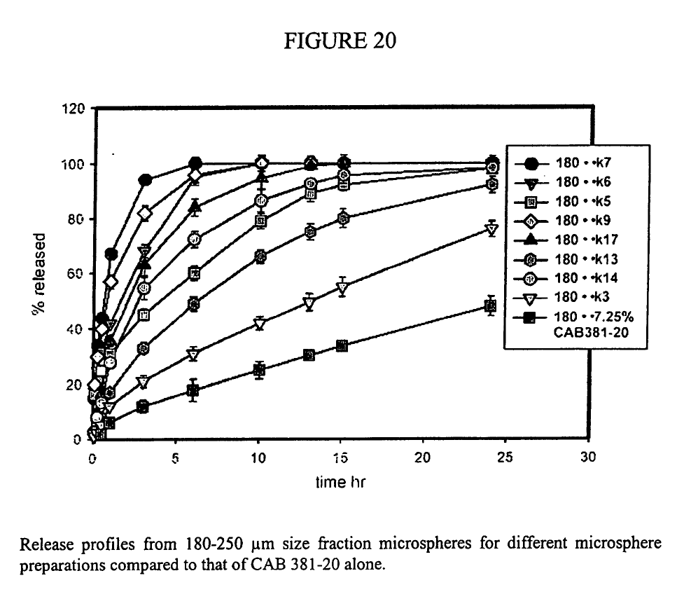

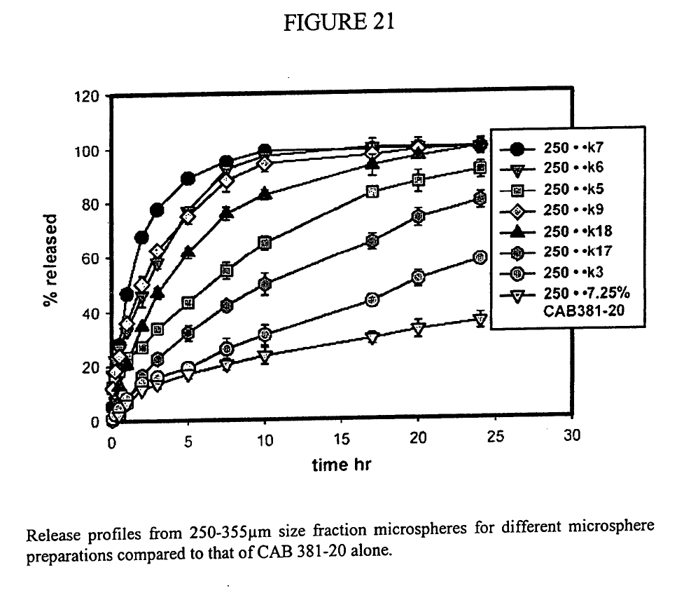

A comparison of the release rates of different microsphere formulation is shown in FIGS. 20 and 21, where release profiles from 180-250 μm and 250-355 μm size fractions microspheres are shown, respectively. It was evident that the viscosity of the initial polymer mixture solutions has a significant effect on release rates and this effect is most obvious when the amount of HPC is kept constant. Viscosity of the polymer solution phase is mainly due to the CAB381-20 polymer that has a higher viscosity as well as higher proportion in the total polymer solution phase. Lower apparent viscosities resulted in faster release rates and with the addition of HPC to the microspheres matrix, release rates increase further. At higher total polymer viscosity, release rates decrease and higher proportions of HPC are required to compensate for the increased viscosity. However, higher viscosities might show higher release rates when the proportion of HPC is increased so much. For example as shown in FIGS. 20 and 21, although formula K7 has higher initial polymer solution viscosity than that of K5, the release rates are higher in the first. This supports the hypothesis that the balance between the viscosity and the HPC concentration no longer holds and the water swelling properties of HPC create high porosity and easy pathways for the drug to be leached out.

The release profile of theophylline in simulated gastric fluid; SGF (pH 1.2) was also conducted for some preparations. It was anticipated that release profiles would be very much similar due to the properties of theophylline, which is neutral. Generally, release profiles in SGF were found to be similar to those in SIF.

The invention is described further in the following examples, which are illustrative and not limiting.

### EXAMPLE 1

### Preparation and Analysis of CAB-CAP Microspheres

CAB-CAP microspheres of the instant invention described herein were prepared and analyzed as follows.

### Experimental

Materials

The materials used were obtained from the following commercial suppliers and used without further purification. Cellulose acetate butyrate (CAB381-20, Eastman Chem. CO. lot. C-2769-B), cellulose acetate phthalate (CAP, Aldrich Chemical Company Lot 06715TG), theophylline (lot. No. 93237, Knoll AG), sorbitan sesquioleate (Arlacel 83, Sigma), acetone, hydrochloric acid, 36.5-38.0% (J. T. Baker Inc., Phillipsburg, N.J.), heptane (GFS Chemicals, Inc., Columbus, Ohio), mineral oil (Ruger Chemical Co. Inc., Irvington, N.J.). methylene chloride, sodium phosphate tribasic and sodium chloride crystals (Fisher Scientific, NJ).

Instruments

Stirrer (Lab. Stirrer LR 4000, Yamato Scientific Co., LTD, Tokyo, Japan), USP Dissolution Apparatus II (Dissolution test system 5100, Distek, Inc., North Brunswick, N.J.), UV Spectrophotometer (Spectronic 2000, Bausch & Lomb, Rochester, N.Y.), Accumet pH meter 5 (Fisher Scientific, NJ), standard sieve series.

Preparation of Microspheres

Microspheres containing micronized anhydrous theophylline of a theoretical concentration of 33.3% were prepared by the non-aqueous emulsion-solvent evaporation method using cellulose acetate butyrate (CAB 381-20, M. W. of 70,000) at concentrations 7.5 to 10% (w/w) of the total polymer concentration dissolved in acetone and cellulose acetate phthalate (CAP, M. W. 42,000) at ratios of 1-5% of the total polymer concentration. Theophylline powder was dispersed in the above mentioned polymer solution mixtures to yield the required drug to polymer ratios. Sorbitan sesquioleate was used as an emulsifying agent. The stirrer consisted of two propellers on a single shaft. Each propeller contained three blades with a diameter of 25 mm. The dispersion system was continuously stirred at a constant adjusted speed (750-1500 rpm) depending on the viscosity of polymer solutions. After the formation of microspheres and the evaporation of solvent, the microspheres were separated from the oil phase, washed with n-heptane and dried at 50° C. At least two identical batches were prepared of each formula.

Particle Size Distribution

Size distributions were evaluated by sieve analysis using a set of standard sieves with openings from 106 to 600 μm. The microspheres were placed at the topmost sieve and tapped by hand. The weight of microspheres retained on each individual sieve was recorded and the microspheres were stored for further characterization.

Drug Loading

Drug content analysis was performed by accurately weighing about 5 mg samples of microspheres in a 10-ml volumetric flask. A mixture of methylene chloride and ethanol at a ratio of 3:1 was added to dissolve the polymer and the drug. Drug concentration was determined specrophotometric analysis at 274-nm wavelength. Although there were small amounts of phthalate containing polymer in the microspheres, there was insignificant interference with the drug analysis.

Dissolution Analysis

In vitro dissolution studies were carried out on the microspheres at 37° C. (±0.5° C.) at 100 rpm with USP dissolution apparatus II using the procedure for enteric-coated products. For the acid stage, an accurately weighed sample of microspheres (25-30 mg) was suspended in the dissolution media consisting of 525 ml of 0.1 N hydrochloric acid without enzymes and dissolution was done for 2 hours. At the end of the 2 hours, 375 ml of 0.1 M tribasic sodium phosphate was added to all dissolution vessels, the pH was adjusted to 7.5 and the dissolution was continued for 24 hours for the buffer stage. Aliquots of dissolution fluid were withdrawn at specified time intervals to assay the released drug spectrophotometrically at 271 nm. Dissolution was carried out for at least 24 hours. Each graphical data point was an average of dissolution data from three samples. Corrections were made for the removal of samples. The interference of polymers with the absorbance is negligible (maximum of 0.002 absorbance units) due to the small amount of phthalate in CAP polymer (phthalic acid absorbance maximum is at 281 nm in phosphate buffer).

### EXAMPLE 2

### Preparation and Analysis of CAB-HPC Microspheres

CAB-HPC microspheres of the instant invention described herein were prepared and analyzed as follows.

### Experimental

Materials

Cellulose acetate butyrate (CAB381-20, Eastman Chem. CO. lot. C-2769-B), hydroxypropyl cellulose (HPC, Scientific Polymer Products Inc., CAT# 402, Lot 1), theophylline (lot. No. 93237, Knoll AG), sorbitan sesquioleate (Arlacel 83, Sigma), acetone (J. T. Baker Inc., Phillipsburg, N.J.), heptane (GFS Chemicals, Inc., Columbus, Ohio), methylene chloride (Fisher Scientific, NJ), mineral oil (Ruger Chemical Co. Inc., Irvington, N.J.). Potassium phosphate monobasic and sodium hydroxide 50% w/w solution (J. T. Baker Inc., Phillipsburg, N.J.).

Instruments

Stirrer (Lab. Stirrer, LR 4000, Yamato Scientific Co., LTD, Tokyo, Japan), USP dissolution apparatus II (dissolution test system 5100, Distek, Inc., North Brunswick, N.J.), UV spectrophotometer (Spectronic 2000, Bausch & Lomb, Rochester, N.Y.), Accumet pH meter 5 (Fisher Scientific, NJ), standard sieves series, viscometer DV-II (Brookfield Engineering Laboratories, Inc. Stoughton, Mass.)

Preparation of Microspheres

Microspheres containing micronized anhydrous theophylline in a theoretical concentration of 33.3% were prepared by the emulsion-solvent evaporation method using the polymers (CAB 381-20, MWT of 70,000) at concentrations 7 to 9% (w/w) of the total polymer concentration dissolved in acetone and (HPC, MWT 100,000) at a ratio of 0.5-5.25% to that of CAB381-20. Theophylline powder was dispersed in the above-mentioned polymer solution mixtures to yield the required drug to polymer ratios. Arlacel 83 was used as an emulsifying agent. The stirrer consisted of two propellers on a single shaft. Each propeller had a diameter of 25 mm and contained three blades. The dispersion system was continuously stirred at a constant adjusted speed (750-1250 rpm) depending on the relative viscosity of polymer solutions. After the formation of microspheres and the evaporation of solvent, the microspheres were separated from the oil phase, washed with n-heptane and dried at 50° C. At least two identical batches were prepared of each formula.

Viscosity of the Polymer Phase

Viscosities of the polymer mixture solutions were determined with a Brookfield digital viscometer using the small sample adapter with spindles No. 18 and No. 21, as appropriate, at 20 rpm.

Particle Size Distribution

The size distributions were evaluated by sieve analysis using a set of standard sieves from 106 to 600 μm. The microspheres were placed at the topmost sieve and tapped by hand. The weight of microspheres retained on each individual sieve was recorded.

Drug Loading

A 5-mg sample of microspheres was placed in a 10-ml volumetric flask and methylene chloride was added to dissolve the polymers and the drug. Drug concentration was determined specrophotometrically at 274-nm wavelength. At the specified wavelength, no spectrophotometric interferences were observed from an equivalent quantity of the blank microspheres (microspheres without theophylline).

In Vitro Drug Dissolution Analysis

In vitro dissolution studies were carried out on the microspheres at 37° C. (±0.5° C.) in 900 ml of simulated intestinal fluid (SIF) USP without enzyme at 100 rpm using USP dissolution apparatus II. Accurately weighed samples of microspheres (25-30 mg) were suspended in the dissolution media and an aliquot of dissolution fluid was withdrawn at specified time intervals to assay the released drug spectrophotometrically at 271 nm. Dissolution was carried out for at least 24 hours. Each graphical data point was an average of dissolution data from three samples. Corrections were made for the removal of samples.

1. Microspheres comprising an active ingredient dispersed within a polymeric composition comprising a first pH insensitive hydrophobic polymer and second pH sensitive hydrophobic polymer, wherein the microspheres, in an aqueous environment having a pH of around 5 or greater, release the active ingredient in a substantially zero-order profile, and wherein:
(a) the microspheres are formed by a non-aqueous emulsion solvent evaporation method in which the first and second polymers and active ingredient are dispersed in an organic solvent to form a polymer solution phase, the polymer solution phase is emulsified into a second continuous phase comprising a second solvent and a surfactant to form an emulsified dispersion system, and the emulsified dispersion system is agitated and organic solvent evaporated therefrom to form the microspheres;
(b) the concentration of the second polymer as a percentage of total polymer in the polymer solution phase ranges from around 1% to 35% and total polymer concentration in the polymer solution phase ranges from around 5% to around 35%;
(c) microsphere particle diameter ranges from approximately 25 μm to approximately 1,000 μm;
(d) the weight percentage of active ingredient in a microsphere ranges from around 5% to around 50%; and
(e) active ingredient concentration is highest in the microsphere core.

2. Microspheres of claim 1, wherein:
(a) the first and second polymers are selected from the group consisting of cross-linked polyvinyl alcohol, polyvinyl chlorides, regenerated insoluble non-erodible cellulose, acylated cellulose, esterified celluloses, cellulose acetate propionate, cellulose acetate butyrate, cellulose acetate phthalate, cellulose acetate diethyl-aminoacetate, polyurethanes, polycarbonates, and microporous polymers formed by co-precipitation of a polycation and a polyanion modified insoluble collagen;
(b) the organic solvent is a halogenated hydrocarbon, an alcohol, acetonitrile, or ketone, or mixture thereof;
(c) the second solvent is silicone oil, sesame oil, soybean oil, corn oil, cottonseed oil, coconut oil, linseed oil, mineral oil, n-hexane, n-heptane, or mixtures thereof; and
(d) the surfactant is an anionic surfactant, nonionic surfactant, polyoxyethylene-castor oil derivative, polyvinylpyrrolidone, polyvinyl alcohol, lecithin, or sorbitan sesquioleate, or mixtures thereof.

3. Microspheres of claim 1, wherein the first and second polymers are selected from the group consisting of cellulose acetate dimethylamino acetate, cellulose acetate ethyl and methyl carbonate, cellulose acetate phthalate, cellulose acetate succinate, cellulose acetate chloroacetate, cellulose diacetate, cellulose triacetate, cellulose acetate ethyl oxalate, cellulose acetate trimellitate, hydroxypropyl methylcellulose phthalate, cellulose acetate methyl and butyl sulfonate, cellulose acetate octate, cellulose acetate laurate, cellulose acetate p-toluene sulfonate, cellulose acetate ethyl carbamate, cellulose acetate methyl carbamate, cellulose acetate valerate, cellulose acetate maleate, and combinations and mixtures thereof.

4. Microspheres of claim 1, wherein the first and second polymers are selected from the group consisting of cellulose acetate, cellulose acetate propionate, cellulose acetate butyrate, cellulose acetate phthalate, cellulose acetate trimellitate, cellulose propionate butyrate, and combinations and mixtures thereof.

5. Microspheres of claim 1, wherein the first polymer is cellulose acetate butyrate (CAB) and the second polymer is cellulose acetate phthalate (CAP).

6. Microspheres of claim 5, wherein the total concentration of polymer in the polymer solution phase is between around 5% to around 15%.

7. Microspheres of claim 6, wherein the total concentration of CAP in the polymer solution phase as a percentage of total polymer is between around 1% to 30%.

8. Microspheres of claim 7, wherein the average microsphere size is 100 μm to 700 μm.

9. Microspheres of claim 8, wherein the total concentration of CAB in the polymer solution phase is between around 7% to around 9%, the total concentration of CAP in the polymer solution phase as a percentage of total polymer is between around 1% to 3%, and the microspheres are 150 μm to approximately 350 μm in size.

10. Microspheres of claim 8, wherein the microspheres comprise around 30% to around 35% by weight of one or more of the following active ingredients: an alpha-adrenergic agonist an analgesic or anti-migraine agent, an anti-allergic agent, an anesthetic agent, an anoretic agent, a anti-bacterial (antibiotic) agent an anti-cancer agent, an anti-cholinergic agents an anti-diabetic agent, an anti-emetic agent, an anti-fungal agent an antihistamine agent, an anti-hyperlipoproteinemic agent, an anti-hyperthyroid agent, an anti-inflammatory or corticoid agent, an anti-malarial agent, an anti-Parkinson's or Anti-Alzheimer's agent, an anti-psychotic, anti-anxiety or anti-depressant agent, an anti-ulcerative agent, an anti-viral agent, an anxiolytic agent, a B-Adrenergic agonist agent, a bronchodilator, a cardioactive agent, a central nervous system stimulant, a cholinergic agent, a muscle relaxant, and a narcotic antagonist agent.

11. Microspheres of claim 5, wherein the organic solvent is acetone, the surfactant is sorbitan sesquioleate, and the active ingredient is theophylline.

12. Microspheres of claim 5, wherein, upon dispersion of the microspheres into an aqueous environment having a pH of around 5 or greater, substantially all of the active ingredient is released from the microspheres in between around 12 to 24 hours.

13. Microspheres of claim 12, wherein substantially all of the second polymer dissolves in the aqueous environment upon release of substantially all of the active ingredient from the microspheres.

14. A controlled-release pharmaceutical composition comprising microspheres of claim 1.

15. A controlled-release pharmaceutical composition of claim 14, wherein the pharmaceutical composition is a tablet.

16. A controlled-release pharmaceutical composition of claim 14, wherein the pharmaceutical composition is administered orally, parenterally, by inhalation spray, topically, rectally, nasally, buccally, vaginally or via an implanted reservoir

17. A controlled-release pharmaceutical composition of claim 14, wherein the pharmaceutical composition is a tablet or capsule which, upon administration to a mammal, releases substantially all of the active ingredient in the mammal's small intestine.

18. Microspheres comprising an active ingredient dispersed within a polymeric composition comprising a first pH insensitive hydrophobic polymer and second water-swellable polymer, wherein the microspheres, in an aqueous environment, release the active ingredient in a substantially zero-order profile, and wherein:
(a) the microspheres are formed by a non-aqueous emulsion solvent evaporation method in which the first and second polymers and active ingredient are dispersed in an organic solvent to form a polymer solution phase, the polymer solution phase is emulsified into a second continuous phase comprising a second solvent and a surfactant to form an emulsified dispersion system, and the emulsified dispersion system is agitated and organic solvent evaporated there from to form the microspheres;
(b) the concentration of the second polymer as a percentage of total polymer in the polymer solution phase ranges from around 0.25% to 10%, total polymer concentration in the polymer solution phase ranges from around 5% to around 35%, and the viscosity of the polymer solution phase ranges from around 20 cps to around 1000 cps, preferably about 50 to about 300 cps;
(c) microsphere particle diameter ranges from approximately 25 μm to approximately 1,000 μm;
(d) the weight percentage of active ingredient in a microsphere ranges from around 5% to around 50%; and
(e) active ingredient concentration is highest in the microsphere core.

19. Microspheres of claim 18, wherein:
(a) the first polymer is selected from the group consisting of cross-linked polyvinyl alcohol, polyolefins, polyvinyl chlorides, cross-linked gelatins, regenerated insoluble non-erodible cellulose, acylated cellulose, esterified celluloses, cellulose acetate propionate, cellulose acetate butyrate, cellulose acetate diethyl-aminoacetate, polyurethanes, polycarbonates, and microporous polymers formed by co-precipitation of a polycation and a polyanion modified insoluble collagen;
(b) the second polymer is selected from the group consisting of a low-substituted cellulose ether or internally cross-linked cellulose derivatives of sodium carboxymethylcellulose, hydroxypropyl-methylcellulose (HPMC), a hydroxypropylcellulose (HPC), a poly(ethylene oxide), an hydroxyethylcellulose, or a hydrogel forming polymer;
(c) the organic solvent is an alcohol, acetonitrile, ketone, or mixture thereof;
(d) the second solvent is silicone oil, sesame oil, soybean oil, corn oil, cottonseed oil, coconut oil, linseed oil, mineral oil, n-hexane, n-heptane, or mixtures thereof; and
(e) the surfactant is an anionic surfactant, nonionic surfactant, polyoxyethylene-castor oil derivative, polyvinylpyrrolidone, polyvinyl alcohol, lecithin, or sorbitan sesquioleate, or mixtures thereof.

20. Microspheres of claim 19, wherein the first polymer is selected from the group consisting of cellulose acetate dimethylamino acetate, cellulose acetate ethyl and methyl carbonate, cellulose acetate succinate, cellulose acetate chloroacetate, cellulose diacetate, cellulose triacetate, cellulose acetate ethyl oxalate, cellulose acetate methyl and butyl sulfonate, cellulose acetate octate, cellulose acetate laurate, cellulose acetate p-toluene sulfonate, cellulose acetate ethyl and methyl carbomate, cellulose acetate valerate, cellulose acetate maleate, and combinations and mixtures thereof.

21. Microspheres of claim 20, wherein the first polymer is selected from the group consisting of cellulose acetate, cellulose acetate propionate, cellulose acetate butyrate, cellulose propionate butyrate, and combinations and mixtures thereof.

22. Microspheres of claim 20, wherein the second polymer is a hydrogel former.

23. Microspheres of claim 18, wherein the first polymer is cellulose acetate butyrate (CAB) and the second polymer is hydroxypropylcellulose (HPC).

24. Microspheres of claim 23, wherein the total concentration of polymer in the polymer solution phase is between around 5% to around 15%.

25. Microspheres of claim 24, wherein the total concentration of HPC in the polymer solution phase as a percentage of total polymer is between around 0.25% to 10%.

26. Microspheres of claim 25, wherein the average microsphere size is 100 μm to 400 μm.

27. Microspheres of claim 26, wherein the total concentration of CAB in the polymer solution phase is between around 7% to around 9%, the total concentration of HPC in the polymer solution phase as a percentage of total polymer is between around 0.5% to 3%, and the microspheres are 150 μm to approximately 350 μm in size.

28. Microspheres of claim 27, wherein the microspheres comprise up to around 35% by weight of one or more of the following active ingredients: an alpha-adrenergic agonist an analgesic or anti-migraine agent, an anti-allergic agent, an anesthetic agent, an anoretic agent, a anti-bacterial (antibiotic) agent an anti-cancer agent, an anti-cholinergic agents an anti-diabetic agent, an anti-emetic agent, an anti-fungal agent an antihistamine agent, an anti-hyperlipoproteinemic agent, an anti-hyperthyroid agent, an anti-inflammatory or corticoid agent, an anti-malarial agent, an anti-Parkinson's or Anti-Alzheimer's agent, an anti-psychotic, anti-anxiety or anti-depressant agent, an anti-ulcerative agent, an anti-viral agent, an anxiolytic agent, a B-Adrenergic agonist agent, a bronchodilator, a cardioactive agent, a central nervous system stimulant, a cholinergic agent, a muscle relaxant, and a narcotic antagonist agent.

29. Microspheres of claim 27, wherein the organic solvent is acetone, the surfactant is Arlacel 83, and the active ingredient is theophylline.

30. Microspheres of claim 23, wherein, upon dissolution in an aqueous environment having a pH of around 5 or 6 or greater, substantially all of the active ingredient is released from the microspheres in between around 12 to 24 hours.

31. A controlled-release pharmaceutical composition comprising microspheres of claim 18.

32. A controlled-release pharmaceutical composition of claim 31, wherein the pharmaceutical composition is a tablet.

33. A controlled-release pharmaceutical composition of claim 31, wherein the pharmaceutical composition is administered orally, parenterally, by inhalation spray, topically, rectally, nasally, buccally, vaginally or via an implanted reservoir.

34. A controlled-release pharmaceutical composition of claim 32, wherein the pharmaceutical composition is a tablet which, upon administration to a mammal, releases substantially all of the active ingredient in the mammal's small intestine.

35. A method of making microspheres comprising:
(a) dispersing a first pH insensitive hydrophobic polymer, a second pH-sensitive hydrophobic polymer, and an active ingredient in an organic solvent to form a polymer solution phase;
(b) emulsifying the polymer solution phase into a second continuous phase comprising a second solvent and a surfactant to form an emulsified dispersion system; and
(c) agitating the emulsified dispersion system and evaporating the organic solvent there from to form the microspheres
wherein (1) the concentration of the second polymer as a percentage of total polymer in the polymer solution phase ranges from around 0.25% to 10% and total polymer concentration in the polymer solution phase ranges from around 5% to around 35% (2) microsphere particle diameter ranges from approximately 25 μm to approximately 1,000 μm (3) the weight percentage of active ingredient in a microsphere ranges from around 5% to around 40%, and (4) active ingredient concentration is highest in the microsphere core.

36. The method of claim 35, wherein:
(a) the first and second polymers are selected from the group consisting of cross-linked polyvinyl alcohol, polyvinyl chlorides, regenerated insoluble non-erodible cellulose, acylated cellulose, esterified celluloses, cellulose acetate propionate, cellulose acetate butyrate, cellulose acetate phthalate, cellulose acetate diethyl-aminoacetate, polyurethanes, polycarbonates, and microporous polymers formed by co-precipitation of a polycation and a polyanion modified insoluble collagen;
(b) the organic solvent is an alcohol, acetonitrile, or ketone, or mixture thereof;
(c) the second solvent is silicone oil, sesame oil, soybean oil, corn oil, cottonseed oil, coconut oil, linseed oil, mineral oil, n-hexane, n-heptane, or mixtures thereof; and
(d) the surfactant is an anionic surfactant, nonionic surfactant, polyoxyethylene-castor oil derivative, polyvinylpyrrolidone, polyvinyl alcohol, carboxymethylcellulose, lecithin, gelatin, hyaluronic acid, or sorbitan sesquioleate, or mixtures thereof.

37. The method of claim 35, wherein the first and second polymers are selected from the group consisting of cellulose acetate dimethylamino acetate, cellulose acetate ethyl and methyl carbonate, cellulose acetate phthalate, cellulose acetate succinate, cellulose acetate chloroacetate, cellulose diacetate, cellulose triacetate, cellulose acetate ethyl oxalate, cellulose acetate methyl and butyl sulfonate, cellulose acetate octate, cellulose acetate laurate, cellulose acetate p-toluene sulfonate, cellulose acetate ethyl and methyl carbomate, cellulose acetate valerate, cellulose acetate maleate, and combinations and mixtures thereof.

38. The method of claim 35, wherein the first and second polymers are selected from the group consisting of cellulose acetate, cellulose acetate propionate, cellulose acetate butyrate, cellulose acetate phthalate, cellulose propionate butyrate, and combinations and mixtures thereof.

39. The method of claim 35, wherein the first polymer is cellulose acetate butyrate (CAB) and the second polymer is cellulose acetate phthalate (CAP).

40. The method of claim 39, wherein the total concentration of polymer in the polymer solution phase is between around 5% to around 15%.

41. The method of claim 40, wherein the total concentration of CAP in the polymer solution phase as a percentage of total polymer is between around 1% to 3%.

42. The method of claim 41, wherein the average microsphere size is 100 μm to 700 μm.

43. The method of claim 42, wherein the total concentration of CAB in the polymer solution phase is between around 7% to around 9%, the total concentration of CAP in the polymer solution phase as a percentage of total polymer is between around 1% to 3%, and the microspheres are 150 μm to approximately 350 μm in size.

44. The method of claim 43, wherein the microspheres comprise up to around 35% by weight of one or more of the following active ingredients: an alpha-adrenergic agonist an analgesic or anti-migraine agent, an anti-allergic agent, an anesthetic agent, an anoretic agent, a anti-bacterial (antibiotic) agent an anti-cancer agent, an anti-cholinergic agents an anti-diabetic agent, an anti-emetic agent, an anti-fungal agent an antihistamine agent, an anti-hyperlipoproteinemic agent, an anti-hyperthyroid agent, an anti-inflammatory or corticoid agent, an anti-malarial agent, an anti-Parkinson's or Anti-Alzheimer's agent, an anti-psychotic, anti-anxiety or anti-depressant agent, an anti-ulcerative agent, an anti-viral agent, an anxiolytic agent, a B-Adrenergic agonist agent, a bronchodilator, a cardioactive agent, a central nervous system stimulant, a cholinergic agent, a muscle relaxant, and a narcotic antagonist agent.

45. The method of claim 43, wherein the organic solvent is acetone, sorbitan sesquioleate is used to emulsify the dispersion system, and the active ingredient is theophylline.

46. The method of claim 43, wherein the microspheres, upon dissolution in an aqueous environment having a pH of around 5 or 6 or greater, substantially all of the active ingredient is released from the microspheres in between around 12 to 24 hours.

47. The method of claim 43, wherein substantially all of the second polymer dissolves in the aqueous environment to release of substantially all of the active ingredient from the microspheres.

48. A method of making microspheres comprising:
(a) dispersing a first pH insensitive hydrophobic polymer, a second water-swellable polymer, and an active ingredient in an organic solvent to form a polymer solution phase;
(b) emulsifying the polymer solution phase into a second continuous phase comprising a second solvent and a surfactant to form an emulsified dispersion system; and
(c) agitating the emulsified dispersion system and evaporating the organic solvent there from to form the microspheres
wherein (1) the concentration of the second polymer as a percentage of total polymer in the polymer solution phase ranges from around 0.25% to 10%, total polymer concentration in the polymer solution phase ranges from around 5% to around 35%, and the viscosity of the polymer solution phase ranges from around 50 cps to around 1000 cps (2) microsphere particle diameter ranges from approximately 25 μm to approximately 1,000 μm (3) the weight percentage of active ingredient in a microsphere ranges from around 5% to around 50%; and (4) active ingredient concentration is highest in the microsphere core.

49. The method of claim 48, wherein:
(a) the first polymer is selected from the group consisting of cross-linked polyvinyl alcohol, polyolefins, polyvinyl chlorides, acylated cellulose, esterified celluloses, cellulose acetate propionate, cellulose acetate butyrate, cellulose acetate diethyl-aminoacetate, polyurethanes, polycarbonates, and microporous polymers formed by co-precipitation of a polycation and a polyanion modified insoluble collagen;
(b) the second polymer is selected from the group consisting of a low-substituted cellulose ether or internally cross-linked cellulose derivatives of sodium carboxymethylcellulose, hydroxypropyl-methylcellulose (HPMC), a hydroxypropylcellulose (HPC), a poly(ethylene oxide), or a hydrogel forming polymer;
(c) the organic solvent is an alcohol, acetonitrile, or ketone, or mixture thereof;
(d) the second solvent is silicone oil, sesame oil, soybean oil, corn oil, cottonseed oil, coconut oil, linseed oil, mineral oil, n-hexane, n-heptane, or mixtures thereof; and
(e) the surfactant is an anionic surfactant, nonionic surfactant, polyoxyethylene-castor oil derivative, polyvinylpyrrolidone, polyvinyl alcohol, lecithin, or sorbitan sesquioleate, or mixtures thereof.

50. The method of claim 48, wherein the first polymer is selected from the group consisting of cellulose acetate dimethylamino acetate, cellulose acetate ethyl and methyl carbonate, cellulose acetate succinate, cellulose acetate chloroacetate, cellulose diacetate, cellulose triacetate, cellulose acetate ethyl oxalate, cellulose acetate methyl and butyl sulfonate, cellulose acetate octate, cellulose acetate laurate, cellulose acetate p-toluene sulfonate, cellulose acetate ethyl and methyl carbamate, cellulose acetate valerate, cellulose acetate maleate, and combinations and mixtures thereof.

51. The method of claim 48, wherein the first polymer is selected from the group consisting of cellulose acetate, cellulose acetate propionate, cellulose acetate butyrate, cellulose acetate phthalate, cellulose propionate butyrate, and combinations and mixtures thereof.

52. The method of claim 48, wherein the second polymer is a hydrogel forming polymer.

53. The method of claim 48, wherein the first polymer is cellulose acetate butyrate (CAB) and the second polymer is hydroxypropylcellulose (HPC).

54. The method of claim 53, wherein the total concentration of polymer in the polymer solution phase is between around 5% to around 35%.

55. The method of claim 54, wherein the total concentration of HPC in the polymer solution phase as a percentage of total polymer is between around 0.25% to 10%.

56. The method of claim 55, wherein the average microsphere size is 100 μm to 400 μm.

57. The method of claim 55, wherein the total concentration of CAB in the polymer solution phase is between around 7% to around 9%, the total concentration of HPC in the polymer solution phase as a percentage of total polymer is between around 0.5% to 3%, and the microspheres are 150 μm to approximately 350 μm in size.

58. The method of claim 55, wherein the microspheres comprise up to around 35% by weight of one or more of the following active ingredients: an alpha-adrenergic agonist an analgesic or anti-migraine agent, an anti-allergic agent, an anesthetic agent, an anoretic agent, a anti-bacterial (antibiotic) agent an anti-cancer agent, an anti-cholinergic agents an anti-diabetic agent, an anti-emetic agent, an anti-fungal agent an antihistamine agent, an anti-hyperlipoproteinemic agent, an anti-hyperthyroid agent, an anti-inflammatory or corticoid agent, an anti-malarial agent, an anti-Parkinson's or Anti-Alzheimer's agent, an anti-psychotic, anti-anxiety or anti-depressant agent, an anti-ulcerative agent, an anti-viral agent, an anxiolytic agent, a B-Adrenergic agonist agent, a bronchodilator, a cardioactive agent, a central nervous system stimulant, a cholinergic agent, a muscle relaxant, and a narcotic antagonist agent.

59. The method of claim 55, wherein the organic solvent is acetone, Arlacel 83 is used to emulsify the dispersion system, and the active ingredient is theophylline.

60. The method of claim 55, wherein, upon dissolution in an aqueous environment having a pH of around 5 or 6 or greater, substantially all of the active ingredient is released from the microspheres in between around 12 to 24 hours.
